(19) United States
(12) Patent Application Publication
(10) Pub. No.: US 2025/0134534 A1
(43) Pub. Date: May 1, 2025

(54) Allograft Preparation Station

(71) Applicant: Stryker Corporation, Portage, MI (US)

(72) Inventors: Jeremy Ming Graul, Denver, CO (US); Kyle Craig Pilgeram, San Jose, CA (US)

(21) Appl. No.: 18/929,722

(22) Filed: Oct. 29, 2024

Related U.S. Application Data
(60) Provisional application No. 63/546,662, filed on Oct. 31, 2023.

(51) Int. Cl. A61B 17/16; A61F 2/28

(52) U.S. Cl. CPC A61B 17/1635; A61F 2002/2835

(57) ABSTRACT

An allograft preparation station includes a frame and a cutting guide. The frame includes a pair of outer members and a pair of inner members receivable in respective outer members of the pair of outer members. The pair of inner members, when coupled to the frame, may be adjusted to grip a bone from opposing sides of the bone. The cutting guide may be separately secured to the bone to guide cutting of the bone in order to extract an allograft from the bone.

### CROSS-REFERENCE TO RELATED APPLICATION

This application claims the benefit of U.S. Provisional Patent App. No. 63/546,662, filed Oct. 31, 2023, the disclosure of which is hereby incorporated by reference herein in its entirety.

### BACKGROUND

A shoulder may degenerate or suffer injury to an extent that requires a repair or reconstruction to restore function. In some cases, such repair or reconstruction may involve the implantation of an allograft to replace lost bone mass and effectively rebuild part of the joint. Various techniques and instrumentation have been developed to carry out such purpose, though such technologies are often cumbersome and difficult to use. For example, holding a donor bone in position to extract an allograft with a desired shape may require the use of several hands. And, existing methods for cutting a portion of the donor bone to extract it for use as an allograft require a significant number of procedural steps and may involve the use of many interconnected instrument components.

Accordingly, a need exists for instrumentation and methods to simplify and improve allograft preparation for joint surgeries including shoulder surgeries.

### BRIEF SUMMARY

The present disclosure relates to systems, kits and methods for allograft and soft tissue preparation for use in surgery, such as shoulder repair surgery. Various instrumentation contemplated by the present disclosure may be used together as an efficient means of achieving this purpose. For example, an allograft preparation station may include a frame and a cutting guide. The frame may be adapted to receive different pairs of internal blocks, including a first pair of internal blocks that may be adjusted to grip a donor bone for extracting a hard tissue allograft and separately, a second pair of internal blocks that may be adjusted to compress or smash soft tissue to flatten it as a preparatory step for use of the soft tissue in surgery. Such configuration is advantageous in that use of the frame to secure the donor bone is easy as a single pair of hands are all that is needed to secure the donor bone to the frame. Further, while the donor bone is gripped via the first pair of internal blocks on the frame, a cutting guide may be separately secured to the donor bone to guide cutting of the donor bone to extract an allograft from the donor bone. In this way, anchorage of the cutting guide to the donor bone is straightforward as it does not rely on adjustment of instrumentation relative to the frame or any other instrumentation. Moreover, many embodiments of the cutting guides contemplated by the present disclosure allow for the performance of all cuts to extract the allograft such that only one cutting guide is required. To the extent a desired allograft shape requires particular cut angles, the single cutting guide may be chosen from among several available cutting guides, where each one provides a unique allograft shape based on cutting through distinct slit angles of the respective cutting guides. In this manner, a user may select the guide with the desired cut angles and then use that as the sole cutting guide. Options may also be made available through the provision of guides with varying spacing between the slits through the guide, such variation allowing for the extraction of allografts having different sizes.

Another feature of the frame is the inclusion of a macro-translation mechanism and a micro-translation mechanism for respective gross and fine control of a spacing between holding blocks of the frame when internal blocks such as the first pair of internal blocks are used, as described above. Such arrangement renders it easier to quickly obtain some compression on a donor bone held by the frame via the macro-translation mechanism, and allows for relatively greater compression via use of the micro-translation mechanism. The micro-translation mechanism may be advantageous for the additional reason that when flattening of soft tissue is desired via use of the second pair of internal blocks, additional compression is possible and easier to obtain through use of such micro-translation mechanism. Such additional compression may provide a more desirable degree of flattening, or smashing, of the soft tissue. Moreover, in some examples, the micro-translation mechanism may be configured to include additional features such as one or more of a torque limiting mechanism, a torque indicator, a force indicator and a pressure indicator. Details of these controls are provided in greater detail in the representative embodiments described throughout the application disclosure.

In a first aspect, the present disclosure relates to a system including a frame and a guide. In a first example of a first embodiment, the system includes a frame for retrieving a donor bone material for implantation in a patient and a guide configured for attachment to the donor bone while the donor bone is gripped by the first and second contoured surfaces. The frame includes a first block, a second block movably coupled to the first block, a first internal block coupled to the first block, the first internal block including a first contoured surface component and a second internal block coupled to the second block, the second internal block including a second contoured surface component. The first and second contoured surface components are shaped to complement respective opposing surfaces of a donor bone to be received between the first and second contoured surfaces. And, the first and second contoured surface components are configured to grip the donor bone when the first and second contoured surface components are moved toward each other to press against the donor bone. The guide includes an end plate with a first end surface slit and a second end surface slit spaced apart from the first end surface slit and a first side plate extending at an angle from the end plate, the first side plate including side edges extending from the end plate to a free end of the first side plate and a side surface slit in between the side edges. When the first and second contoured surfaces grip the donor bone and the guide is fixed to the donor bone, the donor bone remains stable while cuts are made through the slits.

In a second example of the first embodiment, the guide of the first example may be configured such that a portion of at least one of the side edges of the guide is a cutting edge. And, in this arrangement, cutting through the first and second end surface slits defines a thickness dimension of an allograft, cutting through the side surface slit defines a height dimension of the allograft, and cutting along the cutting edge at least partially defines a width dimension of the allograft. In a variation of the second example, the allograft cut in the manner described may be such that cutting through the side surface defines a width dimension of the allograft and cutting along the cutting edge at least partially defines a heigh dimension of the allograft. In this manner, the height and the width of the allograft are interchangeable depending on how a user desires to orient the allograft for implantation in a patient. In a third example of the first embodiment, the guide of the second example may include slits such that the first end surface slit is at an angle relative to the second end surface slit. With the slits arranged this manner, the thickness of the allograft produced by cuts made through the first and second end surface slits tapers along the height of the allograft. As described in greater detail elsewhere in the present application, the angulation and spacing of the slits may be based on allograft shapes and/or sizes designed for universal use, which could include small, medium and large sizes, for example, or in other approaches the slit arrangement for a guide may be based on a specific patient that will receive the allograft, i.e., patient-specific. In a fourth example of the first embodiment, the guide of the system of any one of the first through third examples may include a bone-facing surface on the first side plate that is contoured to complement a surface of the donor bone. In a fifth example of the first embodiment, the guide included in the system of any one of the first through fourth examples may include a second side plate extending from the end plate and from the first side plate, the second side plate being oriented at an angle relative to the end plate and the first side plate.

In a sixth example of the first embodiment, the first block and the second block of the frame of the system of any one of the first through fifth examples may be movably coupled by a pair of rails such that a distance between the first block and the second block is adjustable by translating one of the first block and the second block relative to the pair of rails. In a seventh example of the first embodiment, the first block of the sixth example may include a threaded shaft positioned therethrough, the threaded shaft being oriented along an axis passing through the first internal block and the second internal block. The threaded shaft may include a handle end and a contact end opposite the handle end, wherein the threaded shaft is configured such that when the handle end is rotated, movement of the threaded shaft causes the first internal block to translate relative to the first block and move toward the second internal block. In an eighth example of the first embodiment, the first and second contoured components of any one of the first through seventh examples are made of an elastomeric material, the first and second contoured components being shaped to correspond to a portion of a tibia bone or a radius bone positionable in between the first and second contoured components. The portion of the tibia or radius may be a distal portion. In a ninth example of the first embodiment, the first internal block of any one of the first through eighth examples may be removably coupled to the first block and the second internal block may be removably coupled to the second block. In a tenth example of the first embodiment, the frame of the system of the ninth example may include a third internal block and a fourth internal block. The third internal block may include a cavity and be removably couplable to the first block. The fourth internal block may include a protrusion and be removably couplable to the second block. With these features, when the third and fourth internal blocks are disposed in the respective first and second blocks and the first and second blocks are brought together, an object disposed in the cavity is flattened as the cavity and protrusion press against opposite sides of the object. In an eleventh example of the first embodiment, the system of any one of the first through tenth examples may be configured such that when the frame and the guide are in operative communication with the donor bone, a portion of the donor bone separates an entirety of the frame from an entirety of the guide.

In a twelfth example of the first embodiment, the system of any one of the first through eleventh examples may include an anchor disposable through an opening in the guide, the anchor being configured to fix the guide to the donor bone. In a thirteenth example of the first embodiment, the anchor in the system of the twelfth example is a first pin and the system further comprises a second pin disposable through a second opening in the guide, the first and second pins anchoring the guide to the donor bone. In a fourteenth example of the first embodiment, the guide of the system of the twelfth example includes a first protrusion surrounding the opening, the first protrusion having an upper surface angled relative to a surface of the guide surrounding the opening such that an angulation of the upper surface indicates a direction for insertion of the anchor through the opening. Put another way, the angulation of the upper surface of the protrusion provides visual guidance to a user in that such upper surface is normal to the longitudinal axis of the insertion trajectory of the anchor. In a fifteenth example of the first embodiment, the anchor of the twelfth example is a first pin that includes a shaft with an annular protrusion along a length of the shaft, the annular protrusion being positioned along the shaft to limit an extent that the first pin passes into the donor bone. Specifically, a flat surface on an underside of the annular protrusion of the anchor is advanceable with the anchor until pressed against a complementary flat surface of the upper surface of the protrusion on the guide. When the anchor is properly aligned through the hole, the respective flat surfaces are generally parallel to each other upon contact, indicating that the anchor is aligned along a trajectory normal to the upper surface of the protrusion surrounding the anchor opening.

In a first example of a second embodiment of the first aspect, a system includes a frame adapted to hold a bone and a guide configured for attachment to the bone. The system is configured to facilitate retrieval of bone material, such as donor bone material, for implantation in a patient. The frame of the system includes a first member with a first gripping surface and a second member movably coupled to the first member, the second member including a second gripping surface. The first and second gripping surfaces are adapted to be usable to fix the frame to the bone. Similarly, the first member and the second member are configured to clamp the bone. The guide of the system, configured for attachment to the bone while it is held by the frame, includes an end plate and a side plate. The end plate has an outer surface and a bone-facing surface opposite the outer surface. The end plate also includes a first guide surface. The side plate extends at an angle from the end plate and includes a second guide surface. When the first and second gripping surfaces hold the bone fixed relative to the frame and the guide is fixed relative to the bone, a portion of the bone separates an entirety of the frame from an entirety of the guide, and the bone remains stable while cuts are made along the first guide surface and the second guide surface.

In a second example of the second embodiment, the system of the first example may be arranged such that the bone-facing surface of the end plate may include an end plate central protrusion that is positionable within a first trough on an end surface of the bone. In a third example, the system of the second example may be arranged such that the side plate includes a bone-facing surface with a side plate central protrusion thereon. The side plate central protrusion may be aligned with and offset from opposing outer side surfaces of the side plate and may be sized to be positionable within a second trough on a side surface of the bone when the end plate central protrusion of the end plate is positioned within the first trough on the end surface of the bone.

In a fourth example, the system of any one of the first through third examples of the second embodiment may be arranged such that the first guide surface defines part of a first end plate slit passing through the outer and bone-facing surfaces of the end plate. In a fifth example, the system of the fourth example may include a second end plate slit spaced apart from the first end plate slit, the second end plate slit passing through the outer and bone-facing surfaces of the end plate. In a sixth example, the system of the fifth example may be arranged such that the second guide surface is a portion of an outer side surface of the side plate. The various cutting surfaces of the guide may be configured to function such that cutting through the first and second end plate slits defines a thickness dimension of an allograft, cutting through a side plate slit of the side plate defines a height dimension of the allograft, and cutting along the outer side surface at least partially defines a width dimension of the allograft. In a seventh example, the system of the sixth example may be arranged such that the first end plate slit is at an angle relative to the second end plate slit. The relationship between the first and second end plate slits may be such that the thickness of the allograft produced by cuts made through the first and second end plate slits tapers along the height of the allograft.

In an eighth example, the system of the first example may be arranged such that the first guide surface is an outer side surface of the end plate. In a ninth example, the system of the first example may be arranged such that the side plate includes opposing outer side surfaces extending from the end plate to a free end of the side plate, and the second guide surface defines part of a side plate slit in between the opposing outer side surfaces. In a tenth example, the system of the first example may be arranged such that the second guide surface is an outer side surface of the side plate. In an eleventh example, the system of the first example may be arranged such that the side plate of the guide includes an outer surface opposite the bone-facing surface, the outer surface being generally planar. In a twelfth example, the system of the first example may be arranged such that the side plate is a first side plate and the guide further comprises a second side plate extending from the end plate and from the first side plate, the second side plate being oriented at an angle relative to the end plate and the first side plate.

In a thirteenth example of the second embodiment, the first member and the second member of the frame of the system of any one of the first through twelfth examples may be movably coupled by a pair of rails such that a distance between the first member and the second member is adjustable by translating one of the first member and the second member relative to the pair of rails. In a fourteenth example, the first and second gripping surfaces of any one of the first through thirteenth examples are made of an elastomeric material, the first and second gripping surfaces being shaped to correspond to a portion of the bone. The bone may be a tibia, a radius, or an ilium, among others. In a fifteenth example, the system of any one of the first through fourteenth examples may be arranged such that the first gripping surface and the second gripping surface are shaped to complement respective opposing surfaces of the bone to be received between the first and second gripping surfaces. In this example, when the first and second gripping surfaces press against the bone, the bone is held fixed relative to the frame. In a sixteenth example, the system of any one of the first through fifteenth embodiments may be arranged such that a first inner member of the first member is slidably coupled to a first outer member of the first member, the first gripping surface being on the first inner member, and a second inner member of the second member is slidably coupled to a second outer member of the second member, the second gripping surface being on the second inner member. In a seventeenth example, the frame of the sixteenth example may include an actuatable shaft operatively connected to the second member such that actuation of the actuatable shaft controls translation of the second inner member relative to the second outer member. In an eighteenth example, the system of the sixteenth example may be arranged such that the first inner member is removably coupled to the first outer member and the second inner member is removably coupled to the second outer member. In a nineteenth example, the frame of the system of the eighteenth example may include a third inner member and a fourth inner member. The third inner member may include a cavity and be removably couplable to the first outer member. The fourth inner member may include a protrusion and be removably couplable to the second outer member. With these features, when the third and fourth inner members are disposed in the respective first and second outer members and the first and second outer members are brought together, an object disposed in the cavity is flattened as the cavity and protrusion press against opposite sides of the object.

In a twentieth example of the second embodiment, the system of any one of the first through nineteenth examples may include an anchor disposable through an opening in the guide, the anchor being configured to fix the guide to the bone. In a twenty-first example, the system of the twentieth example may be arranged such that the anchor is a first pin and the system also includes a second pin disposable through a second opening in the guide, the first and second pins anchoring the guide to the bone. In a twenty-second example, the guide of the twentieth example may include a first protrusion surrounding the opening, the first protrusion having an upper surface angled relative to a surface of the guide surrounding the opening such that an angulation of the upper surface of the first protrusion indicates a direction for insertion of the anchor through the opening. In a twenty-third example, the guide of the twentieth example may be arranged such that the anchor is a first pin that includes a shaft with an annular protrusion along a length of the shaft, the annular protrusion being positioned along the shaft to limit an extent that the first pin may be passed into the bone. In any of the aforementioned examples, the bone may be a donor bone.

In a second aspect, the present disclosure relates to a system including a frame, a first removable block set and a second removable block set, both removably receivable in the frame. In a first example of a first embodiment, a system includes a frame with a first block and a second block movably coupled to the first block. The first block includes a first recess and the second block includes a second recess that faces the first recess. The system also includes a first removable block set and a second removable block set. The first removable block set is configured for gripping a donor bone used in a joint repair and includes a first internal block and a second internal block. The first internal block is configured to be removably coupled to the frame through receipt within the first recess and includes a first contoured surface shaped to complement a first outer surface of a bone. The second internal block is configured to be removably coupled to the frame through receipt within the second recess and includes a second contoured surface shaped to complement a second outer surface of the bone. Further, when the first and second internal blocks are received in the respective first and second recesses, one of the first internal block and the second internal block is adjustable relative to the frame to modify a distance between the first internal block and the second internal block. The second removable block set is configured for flattening soft tissue and includes a third internal block and a fourth internal block. The third internal block is configured to be removably coupled to the frame through receipt within the first recess and includes a cavity such that when the third internal block is disposed within the first recess, the cavity faces the second block. The fourth internal block is configured to be removably coupled to the frame through receipt within the second recess and includes a protrusion. When the third and fourth internal blocks are received in the respective first and second recesses, one of the third internal block and the fourth internal block is adjustable relative to the frame to cause compression of a segment of soft tissue disposed in between a surface of the cavity and a surface of the protrusion. In the system of this embodiment, the first recess of the frame is configured to receive one of the first internal block and the third internal block at any one time and the second recess of the second block is configured to receive one of the second internal block and the fourth internal block at any one time.

In a second example of the first embodiment, the first internal block in the system of the first example may include a first base and a first insert that defines the first contoured surface, the first insert being made of an elastomeric material. In a third example of the first embodiment, the first insert of the second example may be made of silicone. In a variation of the third example of the first embodiment, the system of the second example may include a first alternative insert, the first alternative insert having a second contoured surface different from the first contoured surface and being removably couplable to the first base of the first internal block. In a fourth example of the first embodiment, the system of any one of the first through third examples may include an alternative first internal block interchangeable with the first internal block, the alternative first internal block having a third contoured surface different from the first contoured surface. In a fifth example of the first embodiment, the internal blocks of the fourth example may be configured such that the first contoured surface is shaped to complement a first size of a tibia or a first size of a radius and the third contoured surface is shaped to complement a different bone compared to the first contoured surface or a different size of the bone complemented by the first contoured surface. In a sixth example of the first embodiment, the first and second recess of the respective first and second blocks in any one of the first through fifth examples may each include a bottom surface and opposing side surfaces extending from the bottom surface, each side surface having a groove along its length, and wherein the first, second, third and fourth internal blocks each have a tongue on opposing side surfaces such that the respective grooves on each recess receive the respective tongues of one of the internal blocks to form a tongue-and-groove engagement.

In a seventh example of the first embodiment, any one of the first through sixth examples of the system may include a third internal block with a cavity sized to complement the protrusion such that when the third internal block and the fourth internal block are outside of the frame and the protrusion is positioned within the cavity, the third internal block is not rotatable relative to the fourth internal block. In an eighth example of the first embodiment, the fourth internal block of any one of the first through sevenths example may include a flange extending away from the second recess when the fourth internal block is disposed in the recess, the flange indicating an extent to which the protrusion is received in the cavity. In a ninth example of the first embodiment, the first block and the second block of any one of the first through eighth examples may be connected by a pair of rails such that a distance between the first block and the second block is adjustable by translating one of the first block and the second block relative to the pair of rails. In a tenth example of the first embodiment, the second block of any one of the first through ninth examples may include a threaded shaft positioned therethrough, the threaded shaft being oriented along an axis passing through the first recess and the second recess, and the threaded shaft including a handle end and a contact end opposite the handle end, wherein the threaded shaft is configured such that when the handle end is rotated, movement of the threaded shaft causes the second internal block or the fourth internal block coupled to the second block to translate relative to the second block and move toward the first block.

In a second embodiment of the system of the second aspect of the present disclosure, a system may include a system of the second aspect and a guide configured to be attached to the donor bone such that when the guide is attached to the donor bone, the guide is spaced apart from the frame, the guide including a plurality of slits therethrough, the plurality of slits being positioned on the guide such when the guide is attached to the donor bone, resection through the plurality of slits at least partially defines a bone segment to be cut from the donor bone.

In a third aspect, the present disclosure relates to a method of using an allograft preparation station to prepare and retrieve allografts for implantation in a patient in surgery. In a first example of a first embodiment, the method includes: positioning a donor bone in between a first block of a frame and a second block of the frame, the first block including a first contoured surface and the second block including a second contoured surface, the first contoured surface facing the second contoured surface; translating one of the first block and the second block toward the other of the first block and the second block to bring the first and second contoured surfaces of the frame into contact with respective opposing surfaces of the donor bone such that the first and second contoured surfaces hold the donor bone in place, the first and second contoured surfaces being shaped to be complementary to the respective opposing surfaces of the donor bone; securing a cutting guide to the donor bone such that an end plate of the cutting guide faces an end surface of the donor bone and a side plate of the cutting guide that extends from the end plate faces a side surface of the donor bone; cutting a first bone slit through the end surface of the donor bone using a cutting tool positioned through a first plate slit in the end plate; cutting a second bone slit through the end surface using the cutting tool positioned through a second plate slit in the end plate, the second plate slit being offset from the first plate slit; cutting a third bone slit through the side surface using the cutting tool positioned through a third plate slit in the side plate; and cutting a fourth bone slit through the donor bone, the fourth bone slit being at an angle relative to each of the first, second and third bone slits. In the method, a portion of the donor bone defined by the first, second, third and fourth bone slits encompasses a bone segment to be implanted into a joint of the patient.

In a second example of the first embodiment of the method, the method of the first example may include inserting a first internal block into a first recess of the first block and inserting a second internal block into a second recess of the second block, the first contoured surface being on the first internal block and the second contoured surface being on the second internal block. In a third example of the first embodiment, the method of one of the first or second examples may include rotating a threaded shaft on the second block to cause the second internal block to translate toward the first internal block and away from the second block thereby applying additional pressure to the donor bone. In a fourth example of the first embodiment, the method of any one of the first through third examples may be performed with the frame configured such that the first block and the second block are coupled through a pair of rails and the first and second blocks are movable with respect to each other by translating one of the first block and the second block relative to the pair of rails.

In a fifth example of the first embodiment, the method of any one of the first through fourth examples may also include, prior to positioning the donor bone in between the first and second blocks or after extraction of the bone segment: removing the first internal block from the first block and inserting a third internal block; removing the second internal block from the second block and inserting a fourth internal block; placing a soft tissue in a cavity of the third internal block; and translating one of the first block and the second block toward the other of the first block and the second block to bring a protrusion of the fourth internal block toward the cavity of the third internal block, thereby applying pressure to the soft tissue disposed in the cavity. In a sixth example of the first embodiment, the method of any one of the first through fifth examples may include using a side edge of the side plate to cut the fourth bone slit. In a seventh example of the first embodiment, the method of any one of the first through sixth examples may include securing the cutting guide to the donor bone includes pressing the side surface against the donor bone and pressuring a second side surface of the guide against the donor bone, the second side surface extending from the end surface at a different angle from the side surface. In an eighth example of the first embodiment, the method of any one of the first through seventh examples may include a donor bone that is a tibia, radius or ilium.

In a second embodiment of the third aspect, a method of using an allograft preparation station to prepare and retrieve allografts for implantation in a patient in surgery includes: positioning a bone in between a first internal block positioned on a first block of a frame and a second internal block positioned on a second block of the frame; moving one of the first internal block and the second internal block toward the other of the first internal block and the second internal block until a first contoured surface of the first internal block and a second contoured surface of the second internal block both press against the bone to fix the bone in place relative to the frame; removing the first internal block from the first block and the second internal block from the second block; inserting a third internal block into the first block and a fourth internal block into the second block such that the respective internal blocks are movably coupled to the respective blocks; disposing a soft tissue into a cavity within one of the third internal block and the fourth internal block; and moving one of the third internal block and the fourth internal block toward the other of the third internal block and the fourth internal block such that a protrusion on the other of the one of the third internal block and the fourth internal block presses against a first side of the soft tissue and the cavity presses against a second side of the soft tissue.

In a third embodiment of the third aspect, a method of using an allograft preparation station to prepare and retrieve allografts for implantation in a patient in surgery includes: using a frame to hold a bone fixed in place relative to the frame by: positioning a bone in between a first internal block positioned on a first block of a frame and a second internal block positioned on a second block of the frame; and moving one of the first internal block and the second internal block toward the other of the first internal block and the second internal block until a first contoured surface of the first internal block and a second contoured surface of the second internal block both press against the bone to fix the bone in place relative to the frame; and using the frame to flatten soft tissue by: disposing a soft tissue into a cavity within a third internal block before or while the third internal block is positioned on the first block; and moving one of the third internal block and a fourth internal block positioned on the second block toward the other of the third internal block and the fourth internal block such that a protrusion on the fourth internal block presses against a first side of the soft tissue and the cavity presses against a second side of the soft tissue. When the method begins with using the frame to hold the bone fixed, subsequent to the first moving step, the first internal block is removed from the first block and the second internal block is removed from the second block, then a third internal block is inserted into the first block and the fourth internal block is inserted into the second block. And, when the method begins with using the frame to flatten soft tissue, subsequent to the second moving step, the third internal block is removed from the first block and the fourth internal block is removed from the second block, then a first internal block is inserted into the first block and the second internal block is inserted into the second block.

In a first example of a fourth embodiment of the third aspect, a method of retrieving an allograft for implantation in a patient includes: positioning a donor bone in between a first member of a frame and a second member of the frame, the first member including a first contoured surface and the second member including a second contoured surface, the first contoured surface facing the second contoured surface; translating the second member toward the first member to bring the first and second contoured surfaces of the frame into contact with respective opposing surfaces of the donor bone such that the first and second contoured surfaces hold the donor bone in place, the first and second contoured surfaces being shaped to be complementary to the respective opposing surfaces of the donor bone; securing a cutting guide to the donor bone such that an end plate of the cutting guide faces an end surface of the donor bone and a side plate of the cutting guide that extends from the end plate faces a side surface of the donor bone; forming a first bone cut through the end surface of the donor bone using a cutting tool positioned along a first guide surface of the end plate; forming a second bone cut through the side surface of the donor bone using the cutting tool positioned along a second guide surface of the side plate; and forming a third bone cut through the donor bone, the third bone cut being at an angle relative to each of the first and second bone cuts. Through the performance of this method, a portion of the donor bone defined by the first, second and third bone cuts encompasses a bone segment to be implanted into a joint of the patient.

In a second example of the fourth embodiment, the method of the first example may include forming a fourth bone cut through the end surface using the cutting tool positioned through a plate slit in the end plate. In a third example, the method of the first example may include forming a fourth bone cut through the side surface using the cutting tool positioned through a plate slit in the side plate. In a fourth example, the method of the first example may be performed with the first guide surface of the guide defining part of a plate slit in the end plate and such that forming the first bone cut includes using the cutting tool positioned through the plate slit. In a fifth example, the method of the first example may be performed with the first guide surface being an outer side surface of the end plate and such that forming the first bone cut includes using the cutting tool positioned along the outer side surface. In a sixth example, the method of the first example may be performed with the second guide surface of the guide defining part of a plate slit in the side plate and such that forming the second bone cut includes using the cutting tool positioned through the plate slit. In a seventh example, the method of the first example may be performed with the second guide surface being an outer side surface of the side plate and such that forming the second bone cut includes using the cutting tool positioned along the outer side surface.

In an eighth example of the fourth embodiment, the method of any one of the first through seventh examples may include inserting a first inner member of the first member into a first recess of a first outer member of the first member and inserting a second inner member of the second member into a second recess of a second outer member of the second member, the first contoured surface being on the first inner member and the second contoured surface being on the second inner member. In a ninth example, the method of the eighth example may include rotating a threaded shaft through the second outer member to cause the second inner member to translate toward the first inner member and away from the second member thereby applying additional pressure to the donor bone. In a tenth example, the method of any one of the first through ninth examples may be performed such that the first and second members of the frame used in the method are coupled through a pair of rails and the first and second members are movable with respect to each other by translating one of the first member and the second member relative to the pair of rails. In an eleventh example, the method of the eighth example may include, prior to positioning the donor bone in between the first and second members or after extraction of the bone segment: removing the first inner member from the first member and inserting a third inner member; removing the second inner member from the second member and inserting a fourth inner member; placing a soft tissue in a cavity of the third inner member; and translating one of the first member and the second member toward the other of the first member and the second member to bring a protrusion of the fourth inner member toward the cavity of the third inner member, thereby applying pressure to the soft tissue disposed in the cavity.

In a twelfth example of the fourth embodiment, the method of the first example may include using an outer side surface of the side plate to form the third bone cut. In a thirteenth example, the method of the first example is performed such that the side plate is a first side plate and securing the cutting guide to the donor bone includes both pressing the first side plate against the donor bone and pressing a second side plate of the cutting guide against the donor bone, the second side plate extending from the end portion at a different angle relative to the first side plate. In a fourteenth example, the method of any one of the first through thirteenth examples is performed on a bone that is one of a tibia, radius or ilium.

In a fourth aspect, the present disclosure relates to a guide usable to retrieve an allograft from a bone. In a first example of a first embodiment, a guide is configured for retrieving a donor bone material for implantation in a patient. The guide includes an end plate and a side plate. The end plate has an outer surface and a bone-facing surface opposite the outer surface. The end plate also includes a first guide surface. The side plate extends at an angle from the end plate and includes a second guide surface.

In a second example of the first embodiment of the fourth aspect, the first example may be configured such that the bone-facing surface of the end plate includes an end plate central protrusion that is positionable within a first trough on an end surface of the donor bone. In a third example, the guide of the second example may be configured such that the side plate includes a bone-facing surface with a side plate central protrusion thereon. The side plate central protrusion may be aligned with and offset from opposing outer side surfaces of the side plate and may be sized to be positionable within a second trough on a side surface of the donor bone when the end plate central protrusion of the end plate is positioned within the first trough on the end surface of the donor bone.

In a fourth example of the first embodiment of the fourth aspect, the guide may be configured such that the first guide surface defines part of a first end plate slit passing through the outer and bone-facing surfaces of the end plate. In a fifth example, the guide of the fourth example may include a second end plate slit spaced apart from the first end plate slit, the second end plate slit passing through the outer and bone-facing surfaces of the end plate. In one variation of the fifth example, the second end plate slit may define a cutting plane at an angle relative to a cutting plane of the first end plate slit. In a sixth example, the guide of the fifth example may be configured such that the second guide surface is a portion of an outer side surface of the side plate such that cutting through the first and second end plate slits defines a thickness dimension of an allograft, cutting through a side plate slit of the side plate defines a height dimension of the allograft, and cutting along the outer side surface at least partially defines a width dimension of the allograft. In a seventh example, the guide of the sixth example may be configured such that the first end plate slit is at an angle relative to the second end plate slit. The relative angulation of the slits may be such that the thickness of the allograft produced by cuts made through the first and second end plate slits tapers along the height of the allograft. In an eighth example, the guide of the fifth example may be configured such that the side plate includes opposing outer side surfaces extending from the end plate to a free end of the side plate, and the second guide surface defines part of a first side plate slit in between the opposing outer side surfaces. In this example, the side plate may also include a second side plate slit such that a cutting plane of the second side plate slit is orthogonal to a cutting plane of the first side plate slit. In a ninth example, the guide of the eighth example may be configured such that the end plate defines an opening configured to receive a first bone anchor and the side plate defines an opening configured to receive a second bone anchor.

In a tenth example of the first embodiment of the fourth aspect, the guide of any one of the first through third examples may be configured such that the first guide surface is an outer side surface of the end plate. In an eleventh example, the guide of any one of the first through fifth examples may be configured such that the side plate includes opposing outer side surfaces extending from the end plate to a free end of the side plate, and the second guide surface defines part of a side plate slit in between the opposing outer side surfaces. In a twelfth example, the guide of any one of the first through fifth examples may be configured such that the second guide surface is an outer side surface of the side plate. In a thirteenth example, the guide of any one of the first through twelfth examples may be configured such that the side plate of the guide includes a bone-facing surface and an outer surface opposite the bone-facing surface, the outer surface being generally planar. In a fourteenth example, the guide of any one of the first through thirteenth examples may be configured such that the side plate is a first side plate and the guide further comprises a second side plate extending from the end plate and from the first side plate, the second side plate being oriented at an angle relative to the end plate and the first side plate. In a fifteenth example, the guide of any one of the first through fourteenth examples may be configured for use with a tibia, a radius, or an ilium. In a sixteenth example, the guide of any one of the first through fifteenth examples may be configured such that at least one of the end plate and the side plate is configured to be attached directly to the donor bone. In a seventeenth example, the guide of any one of the first through sixteenth examples may define one or more openings, each of the one or more openings being configured to receive one of an anchor or a pin to fix the guide to the donor bone. In an eighteenth example, the guide of the seventeenth example may be configured such that the one or more openings include a first opening configured to receive a first pin and a second opening configured to receive a second pin. In a nineteenth example, the guide of any one of the first through eighteenth examples may be configured such that the end plate and the side plate are formed together monolithically.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1 is a perspective view of a system according to one embodiment of the present disclosure;

FIG. 2 is a perspective view of a step in a method of retrieving and using a donor bone in a surgery, according to one embodiment of the present disclosure;

FIGS. 3-9 are perspective views of respective additional steps in the method of FIG. 2;

FIG. 10 is a close up view of internal blocks adapted for use in a frame, according to one embodiment of the present disclosure;

FIG. 11 is a perspective view of a frame according to one embodiment of the present disclosure;

FIG. 12A is a perspective view of cutting guide according to one embodiment of the present disclosure;

FIG. 12B is a close-up partial sectional view of the cutting guide of FIG. 12A;

FIGS. 13 and 14 are respective side and top views of the cutting guide of FIG. 12;

FIG. 15 is a perspective view of cutting guide according to one embodiment of the present disclosure;

FIGS. 16 and 17 are respective close-up sectional and side views of the cutting guide of FIG. 15;

FIG. 18 is a perspective view of a cutting guide according to one embodiment of the present disclosure;

FIG. 18A is a perspective view of a cutting guide according to one embodiment of the present disclosure;

FIGS. 19 and 20 are respective front and top views of the cutting guide of FIG. 18;

FIG. 21 is a perspective view of a cutting guide according to one embodiment of the present disclosure;

FIG. 22 is a perspective view of a cutting guide according to one embodiment of the present disclosure;

FIG. 23 is a perspective view of the cutting guide of FIG. 22 shown with a section cut;

FIG. 24 is a side view of the cutting guide of FIG. 22;

FIG. 25 is a bottom view of the cutting guide of FIG. 22;

FIG. 26 is an upper rear view of the cutting guide of FIG. 22;

FIGS. 27 and 28 are cross-sectional views of the cutting guide of FIG. 22, with cross-sections being taken through an end plate and a side plate, respectively;

FIG. 28A is a cross-sectional view of the cutting guide of FIG. 22;

FIG. 29 is a perspective view of a frame according to one embodiment of the present disclosure;

FIG. 30 is a side view of the frame of FIG. 29;

FIG. 31 is a bottom view of the frame of FIG. 29;

FIG. 32 is a top view of the frame of FIG. 29, with certain parts shown in phantom;

FIG. 33 is a close-up perspective view of part of the frame of FIG. 29;

FIG. 34 is a perspective view of a frame according to one embodiment of the present disclosure;

FIG. 35 is a perspective view of a frame according to one embodiment of the present disclosure;

FIG. 36 is a perspective view of a frame according to one embodiment of the present disclosure;

FIGS. 37-38 are perspective views of inner member components of the frame of FIG. 36.

### DETAILED DESCRIPTION

As used herein unless stated otherwise, the term “anterior” means toward the front part of the body or the face and the term “posterior” means toward the back of the body. The term “medial” means closer to or toward the midline of the body, and the term “lateral” means further from or away from the midline of the body. The term “inferior” means close to or toward the feet, and the term “superior” means closer to or toward the crown of the head. As used herein, the terms “about,” “approximately,” “generally,” and “substantially” are intended to mean that slight deviations from absolute are included within the scope of the term so modified.

In a first aspect, the present disclosure relates to a system for use in surgical procedures. In some embodiments, the system may be adapted to aid in the extraction of hard tissue from a donor for use as an implant in a patient. As a practical matter, a donor bone as referenced herein is expected to be a bone sourced from a cadaver. The term “donor” is used for ease of reference and to indicate a bone sourced for use as part of an implant, regardless of the context in which the bone is made available. In other examples, the system may be adapted for preparation of donor soft tissue for use in a surgical procedure. In still further embodiments, the system may function for both of the aforementioned purposes: as an aid for hard tissue extraction and for preparation of soft tissue. In the various detailed embodiments described in the present disclosure, the system is a frame system, also referred to simply as a frame.

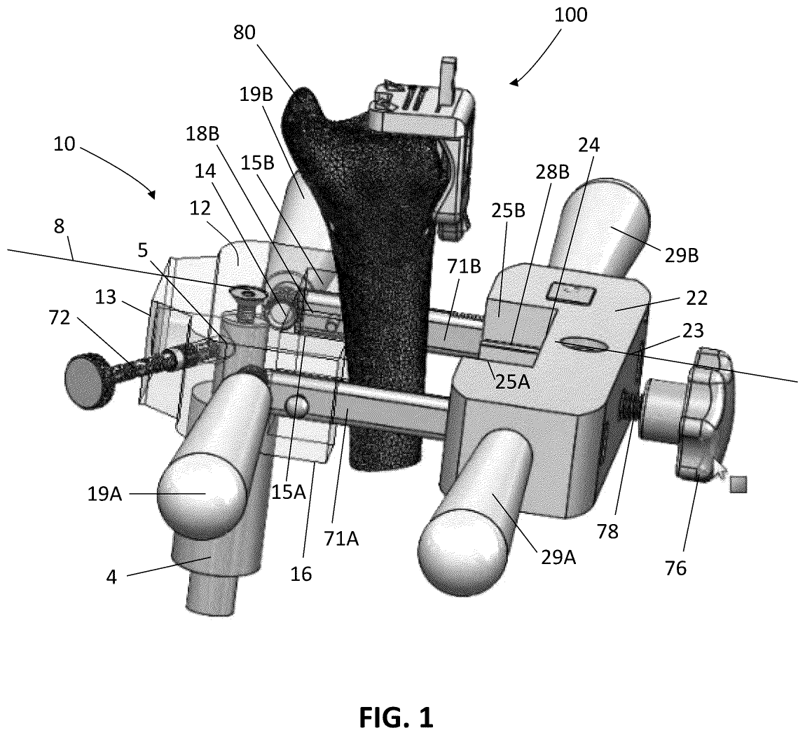

In one embodiment, a frame is indicated by reference numeral 10 and shown in whole or in part in FIGS. 1-5 and 10-11. Frame 10 includes a first block 12 and a second block 22 opposite first block 12, as shown in FIGS. 1-4. First block 12 and second block 22 are coupled to each other through a pair of rails 71A, 71B that extend between the blocks. Rails 71A, 71B are parallel and spaced apart and are received in respective openings within first and second blocks 12, 22 such that first and second blocks 12, 22 may be translated with respect to each other to change a spacing between first and second blocks 12, 22. As shown in the depicted embodiment, each rail includes a plurality of teeth so that an adjustment of spacing between first and second blocks 12, 22 may be made in predetermined increments through interaction with a surface feature or component within at least one of the blocks, such as a pawl on an underside of button 27. As depicted, button 27 may be pressed to control whether the pawl grips the teeth of rails 71A-B and thus whether first and second blocks 12, 22, are movable with respect to each other. In other examples, the rails may have no teeth and an adjustment of spacing between first and second blocks 12, 22 may simply be based on a force applied to one or both of first and second blocks 12, 22, the force being along an axis passing through both blocks 12, 22. First block 12 and second block 22 may also include handles 19A-B, 29A-B as best shown in FIGS. 1 and 3 to aid in the adjustment of spacing between the blocks. First block 12 includes handles 19A, 19B on opposite sides of first block 12, while second block 22 includes handles 29A, 29B on opposite sides of second block 22. It should be appreciated that these handles are optional and may be substituted with other gripping features or may optionally not be included as part of the frame. In another embodiment, first block 12 and second block 22 may be coupled through a hinge connection in place of rails 71A, 71B. Such hinge may be built into one of the blocks in a location proximate a surface where the blocks face each other, or, alternatively, may be an external assembly connected to both blocks on one side of the respective first and second blocks 12, 22, e.g., between handles 19A, 29A. In such embodiment, internal blocks of frame 10, described in greater detail below, may be included in the same manner as in frame 10.

Returning to frame 10, first block 12 also includes components to secure the frame to an external structure, such as a table. One such configuration is shown in FIG. 1, where a support post 4 is disposed through an opening on bottom surface 16 of first block 12. Support post 4 may include an engagement feature such as receiving cavity 5 near an end furthest from a table attachment end to receive a separate locking pin 72. Locking pin 72 may optionally include a shaft with surface characteristics to promote friction when inserted through a designated opening in the first block. In some examples, locking pin 72 may include a feature at its free end that engages with the post at receiving cavity 5. The respective openings in first block 12 to receive support post 4 and locking pin 72 may be orthogonal with respect to each other and enter first block 12 from different sides, but in other examples, the relative angle between the openings may be an acute angle. Further, in additional examples, the features on support post 4 and locking pin 72 that provide engagement between the two may vary. For example, support post 4 may have a protruding feature that is received in a cavity of locking pin 72 proximate a tip of locking pin 72.

Second block 22 includes end surface 23 with an opening (not shown) therein that passes through an entire thickness of second block 22. The opening is aligned with a central longitudinal axis 8 of frame 10, such axis being coincident with a centerline of both first block 12 and second block 22. Disposed through such opening is actuation shaft 78 with a handle 76 attached to an external end thereof. In the depicted embodiment, handle 76 is a knob. Actuation shaft 78 may be threaded and complemented by threading in the second block 22 opening such that translation of the actuation shaft 78 may be controlled through rotation of handle 76. Actuation shaft 78 has sufficient length so that when the handle end of actuation shaft 78 is brought toward end surface 23, an opposite end of actuation shaft 78 protrudes from a central inner surface 24 of second block 22, as shown in FIG. 11. The functionality of this feature is described in greater detail elsewhere in the present disclosure.

Both first block 12 and second block 22 include interior recessed regions as shown in FIG. 1. The recessed region of first block 12 is defined by central inner surface 14 and side surfaces 15A, 15B that are separated by central inner surface 14. Each side surface includes an elongate ridge (one elongate ridge 18B is shown in FIG. 1) that extends generally parallel to central axis 8. Similarly, second block 22 includes a recessed region defined by central inner surface 24 and side surfaces 25A, 25B that are separated by central inner surface 24. Each side surface includes an elongate ridge (one elongate ridge 28B is shown in FIGS. 1 and 11) that extends generally parallel to central axis 8. A shape of the respective recessed regions of frame 10 is generally rectangular, though it is contemplated that this shape may be varied while still preserving the functionality of the frame.

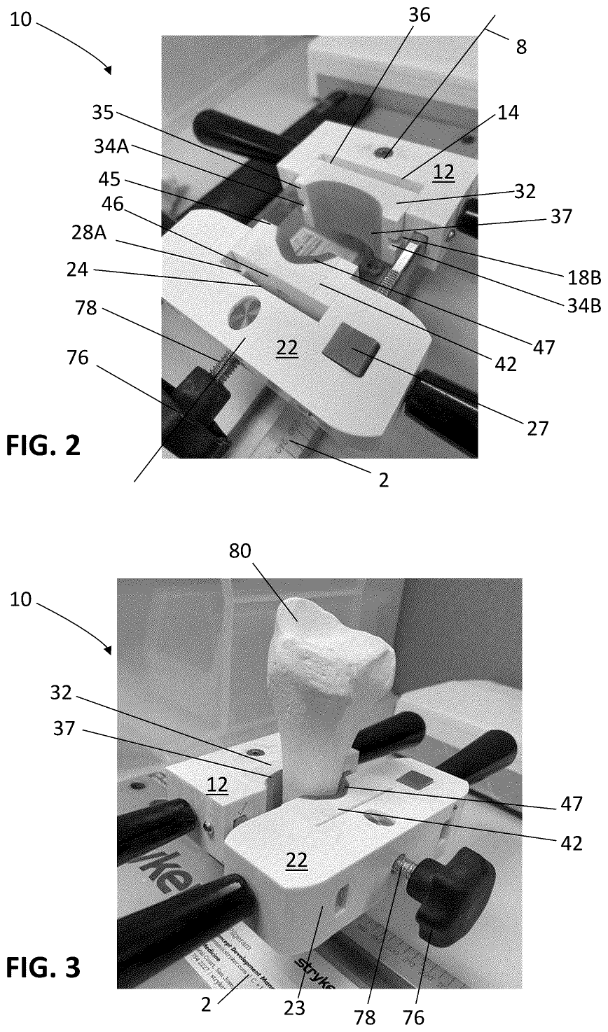

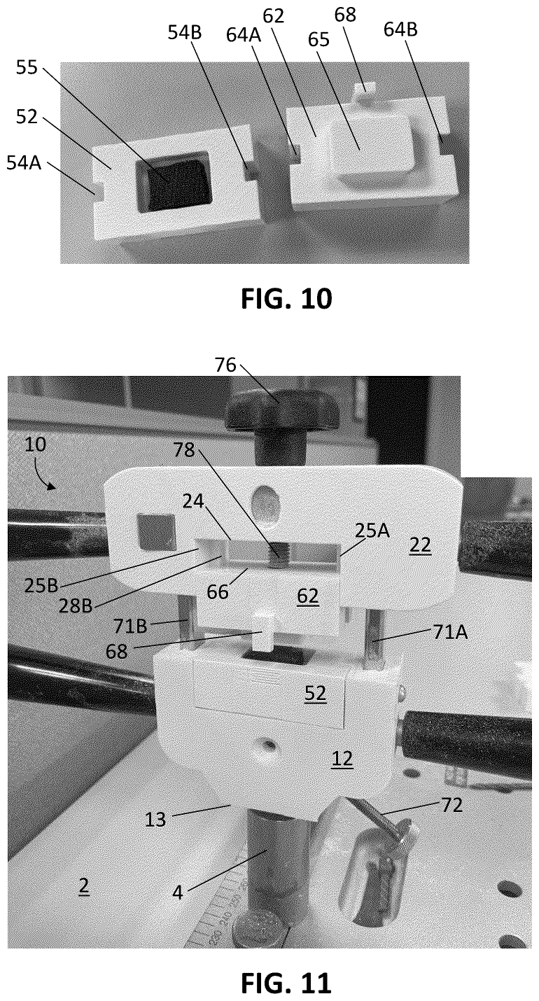

As a system, frame 10 also includes a set of internal blocks 32, 42, 52, 62. First and second internal blocks 32, 42 are shown in FIGS. 2-5 while third and fourth internal blocks 52, 62 are shown in FIGS. 10 and 11. Frame 10 may be arranged in different configurations. In a first configuration, first internal block 32 and second internal block 42 may be received in respective first block 12 and second block 22. In a second configuration, third internal block 52 and fourth internal block 62 may be received in respective first block 12 and second block 22. Details of each configuration will now be discussed in further detail.

In the first configuration, shown in FIGS. 2-4, first internal block 32 is received in first block 12 and second internal block 42 is received in second block 22. Each internal block has an outer surface shaped to be complementary to a recessed region of a respective block of the frame. Specifically, first internal block 32 has a central block-facing surface 36 adapted to face central inner surface 14 of first block 12, and side surfaces of first internal block 32 include respective elongate grooves 34A, 34B. Elongate grooves 34A, 34B are shaped to be slidably received over respective ridges on side walls 15A, 15B of first block 12 to couple first internal block 32 to first block 12. This coupling is a form of a tongue and groove connection. In a variation of the depicted embodiment, respective engagement features on the first internal block and the first block may be different from that shown. In one example, the internal block may have a ridge while the main block may have a groove. In still further variations other complementary sliding mechanisms may be included. Opposite the central block-facing surface, the first internal block includes an engagement surface 35 with a concave portion. The concave portion is oriented such that a trough of the concave portion is orthogonal to central longitudinal axis 8, as shown in FIG. 2.

With continued reference to first internal block 32, disposed on the concave portion of the engagement surface is a first insert 37. First insert 37 has different flexural characteristics compared to a remainder of first internal block 32. In some alternative examples, the first insert may have the same flexural characteristics as first internal block 32. When secured on first internal block 32 while first internal block 32 is coupled to first block 12, first insert 37 has a concave surface facing a center of the frame. A shape including a contour of the concave surface of first insert 37 may be designed to correspond to a particular bone type and a location on the bone type, along with a general size of the bone, e.g., adult or child, etc. One approach for the establishment of a surface shape of first insert 37 is through the aggregation of data from a collection of bones, described in greater detail elsewhere in the present disclosure. Thus, where the bone to be clamped by the frame is a distal tibia, tibial bone data is aggregated to generate a representative design. First insert 37 may be secured to first internal block 32 using securement means as known to persons of ordinary skill in the art. For example, using a protrusion and recess combination between first insert 37 and the first internal block 32 to allow the components to snap together. It should be appreciated that first insert 37 may be substituted with other inserts for purposes of fitting with the donor bone to be gripped by the frame. For example, while first insert 37 may be contoured to fit distal tibia 80, if frame 10 is used to grip a distal radius (not shown), a separate insert with a surface contour more closely conforming to the distal radius may be secured to first internal block 32 in place of first insert 37. Similar principles may also be applied to a single bone type, but for different sizes of such bone type.

Turning to second internal block 42, second internal block 42 generally has the same shape and features as first internal block 32 although has a different concave surface portion configured to face first internal block 32 and is configured to be received in a recessed region of second block 22, as shown in FIG. 2. Similar to first internal block 32, second internal block 42 includes a second insert 47 secured onto the concave surface portion of an engagement surface 45 that faces a center of the frame when second internal block 42 is slidably coupled to second block 22. The above-described variations of the first internal block 32 and first insert 37 are also contemplated for the second internal block 42 and second insert 47, respectively. It should be appreciated that second insert 47 and the surface of second internal block 42 onto which it is received may have a “V” shape with a much smaller radius of curvature than a surface of first insert 37 while also being narrower in width than first insert 37. Such surface contours provide improved surface-to-surface conformance of opposing sides of distal tibia 80 with respective first and second inserts 37, 47. And, in this way, other inserts that may be used in place of those depicted may have different comparative characteristics between them to ensure adequate conformance of the inserts on opposing sides of the donor bone. As with first insert 37, second insert 47 may similarly be designed using data aggregation techniques based on databases. Both first and second inserts 37, 47 may be made of silicone or other similar materials, such as elastomeric materials. Further, and with continued reference to first and second inserts 37, 47, in some variations, one or both of the inserts included on frame 10 may include surface features to promote engagement with a bone received between first and second internal blocks 32, 42. For example, first and second inserts 37, 47 may include protrusions such as ribs or spikes, knurling, or other patterns of surface irregularities to provide additional resistance to movement between first and second inserts 37, 47 and a bone clamped therebetween. Such resistance may include resistance to axial and/or rotational movement of the bone relative to the frame. Optionally, second internal block 42 may include a recess or hole on a central surface 46, central surface 46 being opposite engagement surface 45, where the recess is sized and shaped to be in operative communication with actuation shaft 78 as actuation shaft 78 is actuated through second block 22. This is described in greater detail in the methods of using the contemplated systems.

In the second configuration, shown in FIG. 11, third internal block 52 is slidably disposed into first block 12 and fourth internal block 62 is slidably disposed in second block 22. Third and fourth internal blocks 52, 62 are similar to respective first and second internal blocks 32, 42, with distinctions described in greater detail below. Each internal block has an outer surface shaped to be complementary to a recessed region of a respective block of the frame.

Turning to the individual internal blocks, third internal block 52 has a central block-facing surface adapted to face central inner surface 14 of first block 12, and side surfaces of third internal block 52 include respective elongate grooves 54A, 54B, as shown in FIG. 10. Elongate grooves 54A, 54B are shaped to be slidably received over respective elongate ridges on side walls 15A, 15B of first block 12 to couple third internal block 52 to first block 12. This coupling is a form of a tongue and groove connection. In a variation of the depicted embodiment, respective engagement features on the first internal block and the first block may be different from that shown, with examples of variations including those described with respect to first internal block. Third internal block 52 also includes an engagement surface with a cavity 55 therein, as shown in FIG. 10. Cavity 55 is cuboid in shape with a rectangular cross-section, though other shapes are contemplated. Cavity 55 includes a generally flat recessed surface for receipt of an object such as soft tissue. It should be appreciated that the overall shape of the cavity 55 and a contour of its bottom surface may be varied to any desired shape.

Fourth internal block 62 has the same overall shape as third internal block 52 although is configured to be received in a recessed region of second block 22, as shown in FIG. 11. As with third internal block 52, fourth internal block 62 includes elongate recesses 64A, 64B for receipt over respective elongate ridges on side surfaces 25A, 25B of second block 22. The above-described variations of the engagement features for first internal block 32 are also contemplated for fourth internal block 62. Additionally, fourth internal block 62 includes an engagement surface with a protrusion 65 shaped to complement cavity 55. In the depicted embodiment, protrusion 65 is cuboid and may include chamfers at corner areas. With this structure, when third and fourth internal blocks 52, 62 are coupled to respective first and second blocks 12, 22, and such blocks 12, 22 are closed toward each other, protrusion 65 may advance into cavity 55. Optionally, protrusion 65 may include additional sub-protrusions or other surface irregularities thereon, such as spikes, elongate blades and knurling. The inclusion of additional sub-protrusions may facilitate tenderizing of soft tissue when compressed between the internal blocks, or in other cases, may facilitate the breakup the soft tissue into smaller pieces. Fourth internal block 62 also includes an optional flange 68 with a base on a front surface of block 62 and extending in the same direction from the body of fourth internal block 62 as protrusion 65, as shown in FIG. 10. When protrusion 65 advances into cavity 55, flange 68 is positioned to pass over an outer surface of third internal block 52 and in this way flange 68 does not impede translation between third and fourth internal blocks 52, 62. Flange 68 may include indicators (not shown) such as lines, numbers or other markings to indicate a measurement. Specifically, the indicators may denote an existing space between an end surface of protrusion 65 and a recessed surface of cavity 55. Such measurement may be useful when frame 10 is in use to indicate an extent of compression of material disposed within the cavity 55, as is described in greater detail elsewhere in the present application.

Moreover, as with second internal block 42, fourth internal block 62 may optionally include a recess or hole on a central surface 66, central surface 66 being opposite the engagement surface as shown in FIG. 11. Such recess may be sized and shaped for operative communication with actuation shaft 78 as actuation shaft 78 is actuated through second block 22. This operation is described in greater detail in the methods of using the contemplated systems and kits.

In some variations of frame 10, actuation shaft 78 used to control movement of fourth internal block 62 relative to second block 22, as shown in FIG. 11, may be further complemented with additional features to enhance the process of compressing soft tissue between the third and fourth internal blocks 52, 62. For example, a torque indicator may be operatively connected to the actuation shaft to indicate an amount of torque applied to the actuation shaft 78. In other examples, a stop may be affixed to the actuation shaft to set a maximum extent of advancement of the actuation shaft 78 into the cavity of second block 22, thereby limiting an extent to which fourth internal block 62 may advance toward and onto a soft tissue within third internal block 52. This, in effect, controls a minimum thickness that may result from compression of the soft tissue. In yet another example, the actuation shaft may be operatively connected to a haptic torque limiter. In one specific example, such haptic torque limiter may take the form of a knob with rotatable settings, each corresponding to a particular amount of compression and/or soft tissue thickness for a soft tissue disposed within third internal block 52. In still further examples, the actuation shaft 78 may be complemented by a force indicator and/or a pressure indicator that is operatively connected to actuation shaft 78. It should further be appreciated that these design variations may also be employed and adapted for use with first and second internal blocks 32, 42 in applications where a donor bone is clamped by frame 10.

With continued reference to frame 10, it should be appreciated that support post 4 may be adjusted when changing a setup of the frame between the first configuration and the second configuration. Specifically, while first and second internal blocks 32, 42 are used, support post 4 is received through an opening in first block 12 that extends from a bottom surface 16 of first block 12, as shown in FIG. 1. When support post 4 is secured to a rigid structure such as table 2 below frame 10, this results in frame orientation such that a body of the frame, i.e., central axis 8, is parallel to table 2. In contrast, when third and fourth internal blocks 52, 62 are used, support post 4 is disposed through an opening in outside surface 13 of first block 12, as shown in FIG. 11, while locking pin 72 continues to be used to secure support post 4 in place. In the second configuration, frame 10 is oriented away from a rigid support surface, e.g., table 2, such that central longitudinal axis 8 through a body of frame 10 is orthogonal to table 2.

In other embodiments, the base frame including first and second blocks 12, 22 may be complemented by only the first and second internal blocks 32, 42. In still further embodiments, the frame including first and second blocks 12, 22 may be complemented by only the third and fourth internal blocks 52, 62. In still further embodiments, alternative systems are contemplated where the system includes a frame and two or more pairs of blocks where each pair of internal blocks has different surface contours on its respective engagement surfaces. Thus, for example, a system may include a frame, a first pair of internal blocks with engagement surfaces for a distal tibia, and a second pair of internal blocks with engagement surfaces for a much different bone. In these alternatives, these additional pairs of internal blocks would be in addition to having different sets of first and second inserts 37, 47 for different bone shapes as part of the system. Such a system may be advantageous when there is a significant difference in bone shapes for the bone types expected to be used with the system.

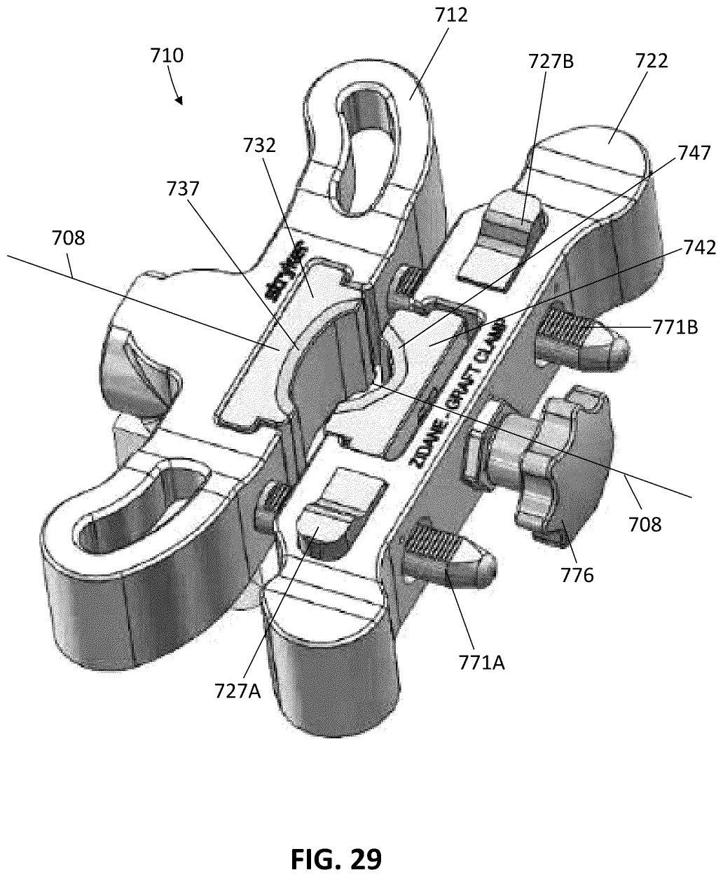

In another embodiment, a frame 710 is shown in FIGS. 29-33. Unless otherwise indicated, like reference numerals refer to like elements of frame 10 shown in FIGS. 1-4 and 10-11, but within the 700 series of numerals. Frame 710 includes a first outer member 712 and a second outer member 722 coupled to first outer member 712 via a pair of rails 771A, 771B. Second outer member 722 is slidable relative to first outer member 712 along axis 708. In the depicted arrangement, a first pawl 727A and a second pawl 727B are operatively disposed within second outer member 722. Each pawl is actuatable to control locking of the second outer member 722 relative to first outer member 712, i.e., through pressing a button of the pawl. Further, rails 771A, 771B as shown include a series of teeth so that adjustment of second outer member 722 may be in predetermined increments.

On second outer member 722, and with reference to FIGS. 29-33, there is an actuation shaft 778 partially disposed within second outer member 722. At an internal end of actuation shaft 778 is a head 779 centered with actuation shaft 778 but having a larger diameter. At an opposite, external end of actuation shaft 778 is a handle 776 fixed to actuation shaft 778. And, along a length of actuation shaft 778 between the ends is a nut 777 disposed over actuation shaft 778. The nut 777 includes internal threading (not shown) to complement threading on actuation shaft 778, and in this way, rotation of the knob controls how far into the second outer member 722 actuation shaft 778 is driven.

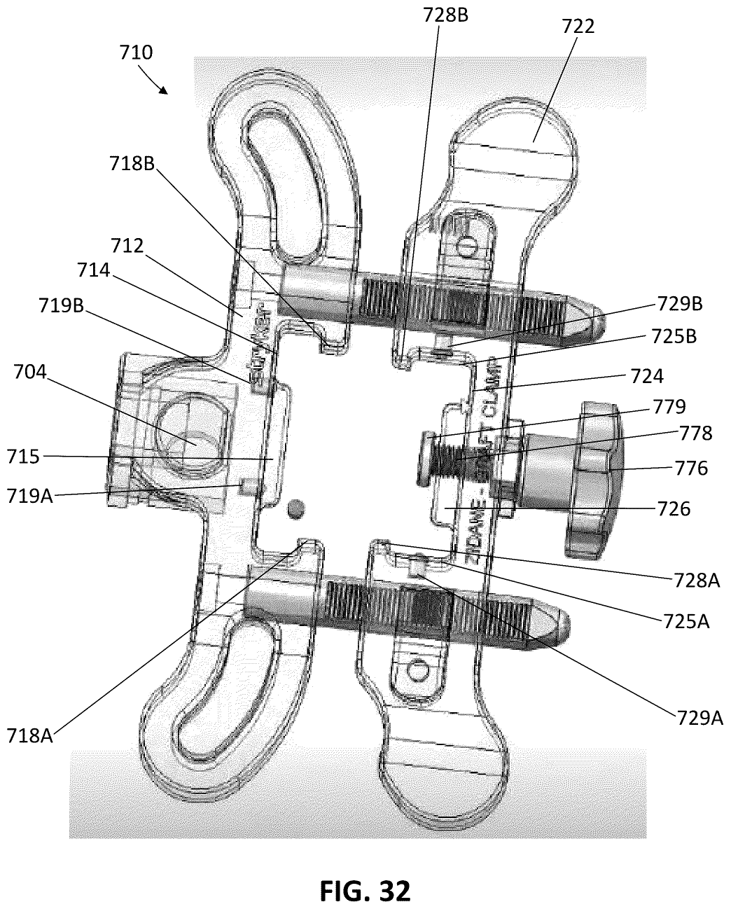

Frame 710 also includes features to provide for secure receipt of first and second inner members 732, 742. Specifically, and with reference to FIG. 32, first outer member 712 includes a recessed region with a central inner surface 714. Extending into the recessed region and spaced apart from an upper surface of first outer member 712 is a ledge 715. Also on central inner surface 714 are spring-loaded grippers 719A, 719B, which are biased to have an extended shape so that each spring-loaded gripper protrudes from central inner surface 714 when not subject to a compression force. In one example, a spring-loaded gripper may be a spring with a solid piece at a free end extending into a recessed region of one of the first and second outer members 712, 722. Similarly, second outer member 722 includes a recessed region defined by a central inner surface 724 and side surfaces 725A, 725B separated by central inner surface 724, again, as shown in FIG. 32. Extending into the recessed region and spaced apart from an upper surface of second outer member 722 is a ledge 726. And, on respective side surfaces 725A, 725B are spring loaded grippers 729A, 729B, configured similarly to those described for first outer member 712. In variations, a different quantity and/or location of spring-loaded grippers may be included as part of the frame.

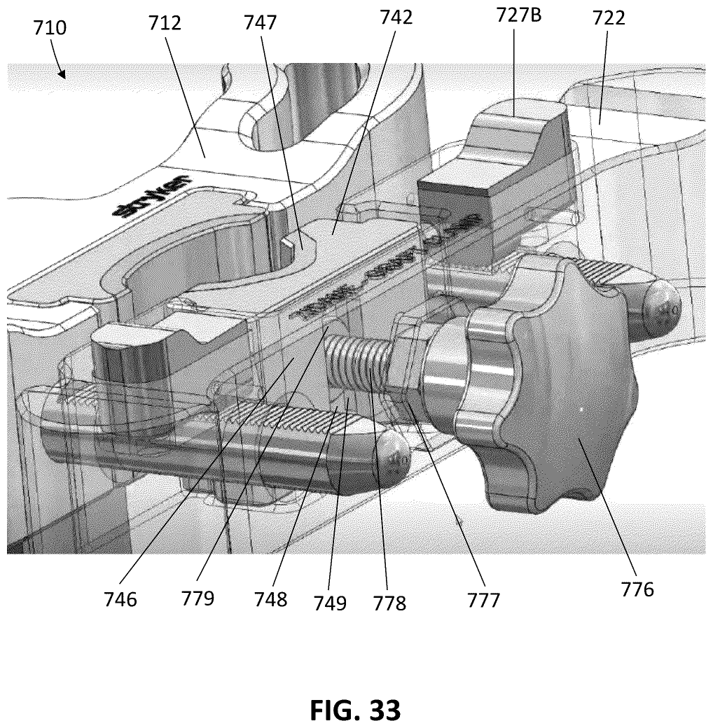

Turning now to first inner member 732 and second inner member 742, each is a standalone component that may be received in the respective first outer member 712 and second outer member 722. First inner member 732 includes a first central surface with a concave contour and a second central surface opposite the first that is slightly wider, as shown in FIG. 29, so that first inner member 732 fits within the recessed region of first outer member 712. The concave surface on the first central surface of first inner member 732 is adapted to receive first gripping member 737. Second inner member 742 includes a first central surface that is concave, as with the first inner member, however, having a smaller radius of curvature that defines more of a V-shape. The concave surface on the first central surface of second inner member 742 is adapted to receive second gripping member 747. Both first and second gripping members 737, 747 may be made of silicone or other similar materials. As shown in FIG. 33, and with continued reference to second inner member 742, second central surface 746 opposite from first central surface includes a recess 748. Recess 748 is shaped to be closed internally within second inner member 742 proximate a top surface of second inner member 742, but extends entirely to a bottom surface edge at an opposite end. Further, within recess 748 is an undercut 749 on both sides of recess 748, so that a width of the opening is wider below the surface level recess 748.

While further detail on the use of frame 710 is addressed elsewhere in the present disclosure, the interaction of the first and second inner members 732, 742 with first and second outer members 712, 722 is briefly summarized here. When first inner member 732 is received in the recessed region of first outer member 712, first inner member 732 is advanceable until it makes contact with ledge 715. As first inner member 732 passes spring-loaded grippers 719A-B, such grippers compress inward. When advancement of first inner member 732 is complete, as shown in FIG. 29, the bias of spring-loaded grippers 719A-B causes them to press against the second central surface of first inner member 732, thereby holding it in place. Specifically, while spring-loaded grippers 719A-B press against second central surface, peripheral surfaces opposite the second central surface press against projections 718A-B on a periphery of recessed region of first outer member 712. When second inner member 742 is received in second outer member 722, actuation shaft 778 is at least partially withdrawn so that undercut 749 of second inner member 742 receives head 779, and at the same time, sides of second inner member 742 are inside of the projections 728A-B at a periphery of the recessed region of second outer member 722. Advancement may continue until second inner member 742 contacts ledge 726. Once in position, spring loaded grippers 729A-B press against side surfaces of second inner member 742 to maintain its position. In some variations, one or both of first and second inner members 732, 742 may include a step or steps on their outer surfaces such that when the applicable inner member is fully received in the outer member, the spring-loaded grippers pop out of the outer members and extend over such step or steps, thereby blocking the inner members from withdrawing from the outer members. Such arrangement provides an additional form of securement to keep the inner member from withdrawing from the outer member.

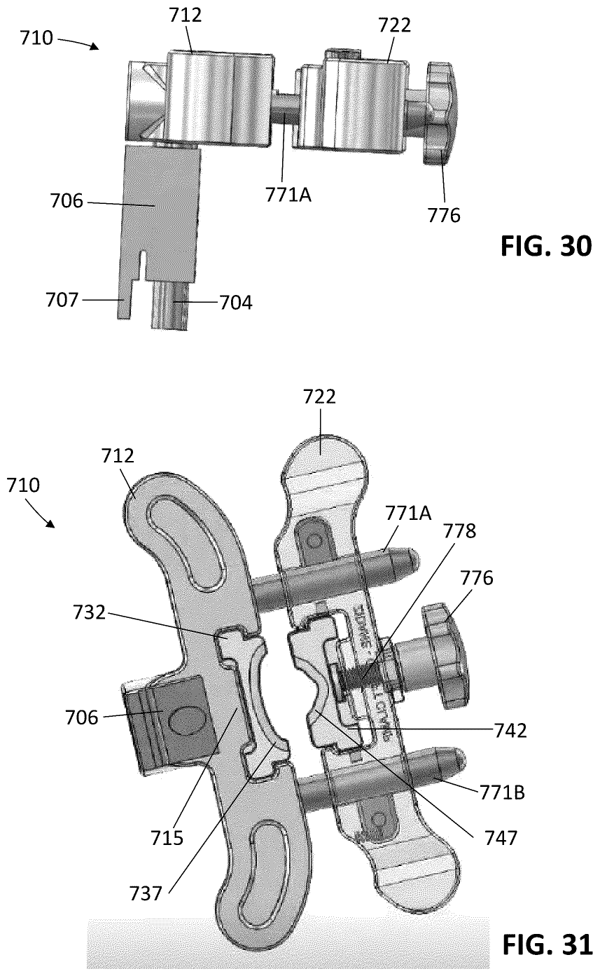

Frame 710 also includes post 704, shown in FIG. 30, that is receivable in first outer member 712. Post 704 may have a “D” shaped cross-section or another similar shape that, when received in the first outer member, prevents the first outer member 712 from rotating relative to post 704. The shape of post 704 is best shown in FIG. 32. While one end of post 704 is receivable in first outer member 712, an opposite end of post 704 may be receivable in a fixed support, such as a table. Further, a sleeve 706 may be received over post 704, as shown in FIG. 30. Sleeve 706 may provide additional anchorage support for post 704 through the inclusion of extension 707, which extends from a body of sleeve 706 and may function as a living hinge. When frame 710 is anchored to a table, extension 707 may be pulled outward from its biased position, increasing a gap between the extension 707 and the body of sleeve 706, to clip the sleeve onto a fixed support, such as a table. When released, extension 707 snaps back and grips the fixed surface, creating a rigid support for frame 710.

In some alternative embodiments, frame 710 may also include third and fourth inner members (not shown) having similar features to third and fourth internal blocks 52, 62 shown in FIGS. 10 and 11, where such third and fourth inner members are compatible with and receivable in respective first and second outer members 712, 722.

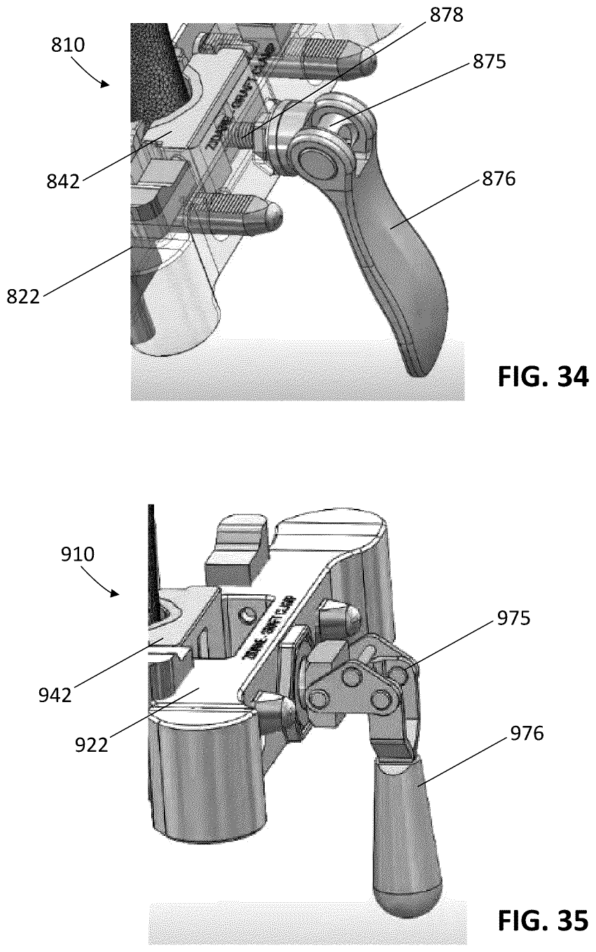

In still further embodiments, frame 710 may include a different micro-translation mechanism in place of actuation shaft 778 and handle 776. One example of this is frame 810 shown in FIG. 34. Unless otherwise indicated, like reference numerals refer to like elements of frame 710 shown in FIGS. 29-33, but within the 800-series of numerals. Second outer member 822 includes actuation shaft 878 disposed therethrough, with a handle 876 attached at an outer end. The handle 876 is rotatable about a hinge connection 875. Rotation of handle 876 controls an extent to which actuation shaft 878 is advanced through second outer member 822, and therefore controls the translation of second inner member 842 relative to second outer member 822. This mechanism may be characterized as a cam handle mechanism. Another example variation of frame 710 is frame 910 shown in FIG. 35. Unless otherwise indicated, like reference numerals refer to like elements of frame 710 shown in FIGS. 29-33, but within the 900-series of numerals. An actuation shaft (not shown) passes through second outer member 922 and at an outer end is connected to handle 976. Handle is rotatable about a hinge mechanism 975 to control axial translation of the actuation shaft, and thus the relative position of second inner member 942 relative to second outer member 922.

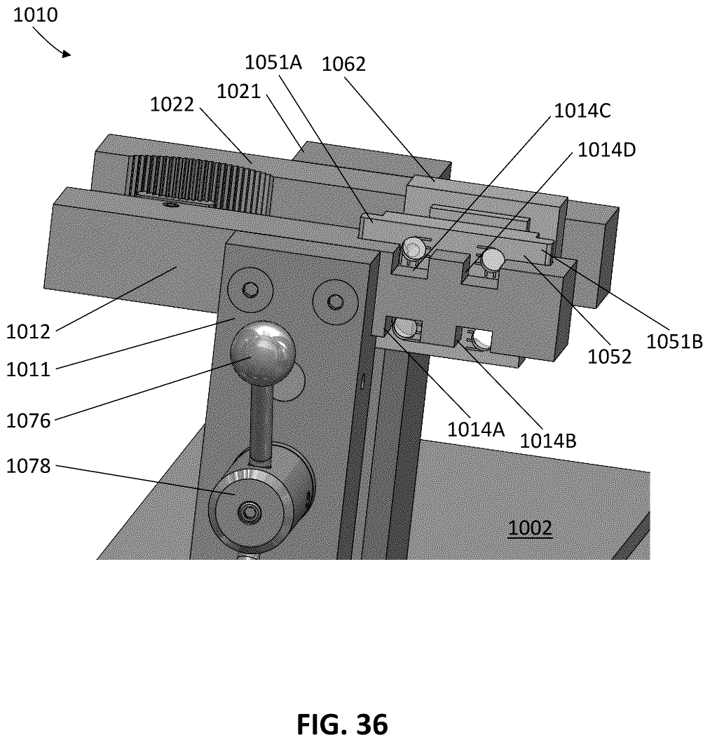

In another embodiment, a frame 1010 is shown in FIG. 36. Unless otherwise indicated, like reference numerals refer to like elements of frame 10 shown in FIGS. 1-4 and 10-11, but within the 1000 series of numerals. Frame 1010 includes a base structure, outer members attached to the base and inner members attached to the outer members. The base structure includes a first base 1011 and a second base 1021. A first outer member 1012 is mounted on first base 1011 and a second outer member 1022 is mounted on second base 1021. The respective outer members 1012, 1022 may be attached to respective first and second bases 1011, 1021 through fasteners, adhesives, welds or other known means. In some examples, the outer member 1012, 1022 may be monolithic with the base 1011, 1021. First outer member 1012 is spaced apart from second outer member 1022 but in general alignment, as shown in FIG. 36. First outer member 1012 may include one or more recesses to allow for visualization through a thickness of first outer member 1012. For example, in the depicted embodiment, first outer member 1012 includes four recesses 1014A-D, on opposite sides of a depth dimension of first outer member 1012. This number may be varied based on the number of windows (e.g., windows 1031A-D) on a first inner member 1052, described in greater detail below. In some examples, a single recess may be large enough to view two or more windows. And, in some examples, there may be no recesses in first outer member 1012. In a subset of such examples, first outer member 1052 may be transparent or translucent to allow for visualization through first outer member 1012.

As to the relative positions of the outer members 1012, 1022, each base 1011, 1021 is operatively connected through a compression control mechanism. As depicted, the compression control mechanism includes an actuation shaft 1078 with a rotatable handle 1076. Actuation shaft 1078 extends between first base 1012 and second base 1022 and may include a threaded shaft (not shown) to mate with a complementary thread within one or both bases 1012, 1022. Rotation of handle 1076 causes a spacing between first outer member 1012 and second outer member 1022 to become closer or further apart, depending on the direction of rotation. First inner member 1052 is attached to first outer member 1012 and second inner member 1062 is attached to second outer member 1022 in a position directly facing first inner member 1052. As depicted, an axis extending through centers of first and second inner members 1052, 1062 is offset from a plane through centers of bases 1011, 1012 and actuation shaft 1078. The inner members 1052, 1062 may be attached to respective outer members 1012, 1022 via fasteners, adhesives, dovetail connections, welds or other similar known means. For first inner member 1052 as depicted, wings 1051A, 1051B on respective lateral sides of first inner member 1052 are received in slots of first outer member 1012.

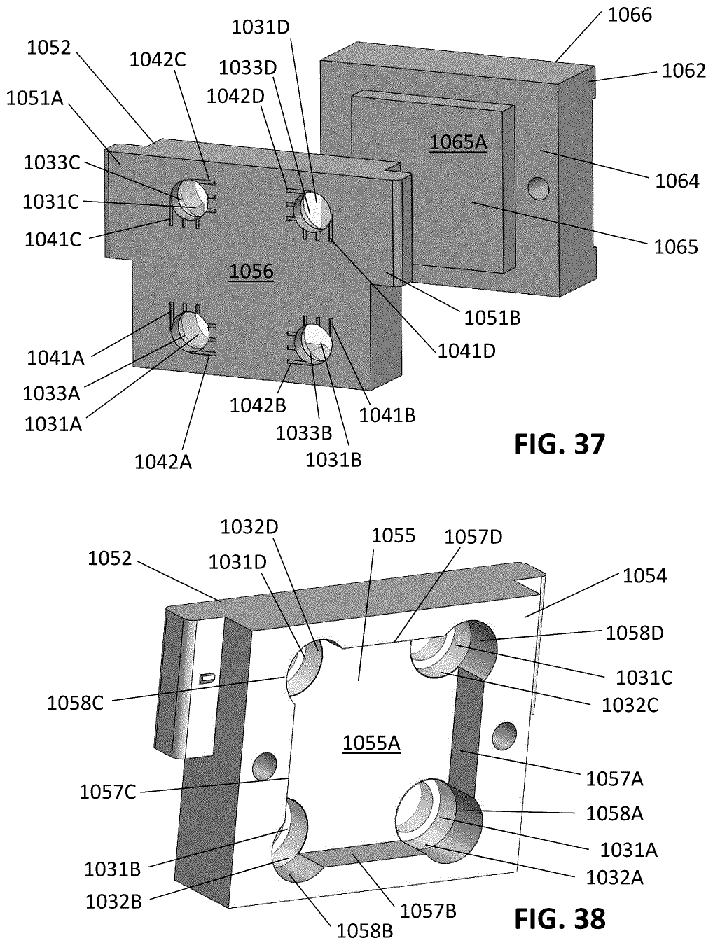

Turning to the details of the first and second inner members, first and second inner members 1052, 1062 are shown in isolation in FIGS. 37-38. First inner member 1052 is complementary to second inner member 1062 in that first inner member 1052 includes a cavity 1055 sized to fit over protrusion 1065 of second inner member 1062. In this manner, an object such as soft tissue placed into a space between cavity surface 1055A of cavity 1055 and surface 1065A of protrusion 1065 may be compressed through the approximation of respective first and second inner members 1052, 1062.

First inner member 1052 includes an inner surface 1054 and an outer surface 1056 opposite inner surface 1054. Inner and outer surfaces 1054, 1056 are generally rectangular as depicted, though in variations, may have any number of other shapes. For instance, according to some exemplary aspects, the first and second inner surfaces 1054, 1056 may be circular or generally circular. Further, surfaces on second inner member 1062 that are complementary to inner and outer surfaces 1054, 1056 may have shapes to match those of first inner member 1052. First inner member 1052 may optionally include first and second wings 1051A-B, as shown. Such wings may be sized to interact with complementary surface features in frame 1010 to hold first inner member 1052 on frame 1010.

Inner surface 1054 of first inner member 1052 defines a cavity 1055 therein, as shown in FIG. 38. First inner member 1052 is also shaped to allow for visualization through a thickness of first inner member 1052. Specifically, first inner member 1052 defines two or more openings therethrough for the receipt of visualization elements so that cavity 1055 is visible from a vantage point external to the outer surface 1056 of first inner member 1052. As depicted in FIGS. 37 and 38, there are four such visualization elements in the form of windows 1031A-D on first inner member 1052. In variations, a first inner member 1052 may include two, three or more visualization elements. Further, a shape of such visualization elements may be cylindrical, cuboid, or any other shape. The openings receiving the respective windows 1031A-D extend from cavity surface 1055A at one end to outer surface 1056 at an opposite end. Each window 1031A-D may be positioned within a respective opening so that an inner window surface 1032A-D is flush with cavity surface 1055A and an outer window surface 1033A-D is recessed relative to outer surface 1056. Recesses on outer surface 1056 are optional. Windows 1031A-D may be secured in place within first inner member 1052 using an adhesive, a mechanical anchorage, or other similar means. Each window 1031A-D is formed of a clear material, i.e., a material that is transparent or translucent. In one example, windows 1031A-D are formed of acrylic material. In the depicted embodiment, a position of windows 1031A-D on first inner member 1052 may be such that part of the respective windows is located outside of a perimeter defined by cavity 1055, as shown in FIG. 38. To provide maximum visualization, walls that define cavity 1055 may include rounded walls 1058A-D that increase a volume of cavity 1055 relative to a volume that would otherwise be defined by cavity walls 1057A-D. Rounded walls 1058A-D are defined at corners of cavity 1055 where walls 1057A-D would otherwise meet, thereby ensuring that no part of a body of first inner member 1052 obstructs visualization through windows 1031A-D. In variations, windows may be positioned more centrally relative to an outer periphery of cavity and in such cases, the first inner member may optionally not include rounded walls to define the cavity.

On outer surface 1056 of first inner member 1052, sets of size markers may optionally be included. In one example, and as depicted, sets of size markers 1041A-D, 1042A-D are positioned along an edge of each respective window 1031A-D. Sets of size markers 1041A, 1042A are representative of the other sets of size markers 1041B-D, 1042B-D, and thus the description for sets of size markers 1041A, 1042A below may apply in the same way for the other sets of size markers. Set of size markers 1041A includes spaced apart lines across a first space adjacent to window 1031A while set of size markers 1042A includes spaced apart lines across a second space adjacent to window 1031A, where set of size markers 1042A are arranged in a direction perpendicular to set of size markers 1041A. The lines of the sets of size markers may be defined by an imprint on outer surface 1056, e.g., ink, may be carved into outer surface 1056, or may even be attached to outer surface through an adhesive. Each set of size markers 1041A, 1042A may include a plurality of markers. As depicted in FIG. 37, each set includes three markers. In other examples, any number of markers may be included in a set. As described in greater detail in the methods of the present disclosure, when a soft tissue is compressed in between first inner member 1052 and second inner member 1062, sets of size markers 1041A, 1042A may be used to visualize an expansion of the soft tissue visible through the windows 1031A-D, thereby providing an indication of the compression of the soft tissue, i.e., with increased compression, a surface area of the soft tissue increases. In variations, the sets of size markers may have a form other than line markers.

As to second inner member 1062, second inner member 1062 includes an inner surface 1064 opposite an outer surface 1066. Protrusion 1065 is a raised surface on inner surface 1064, as shown in FIG. 37. Protrusion 1065 may be shaped to complement a shape of cavity 1055 of first inner member 1052, though will have an at least marginally smaller outer perimeter size so that protrusion 1065 fits within cavity 1055. In some variations, a shape of protrusion 1065 may have some variation relative to that of cavity 1055. In some examples, an elevation of protrusion 1065 relative to inner surface 1064 may be equal to or greater than a depth of cavity 1055, i.e., a depth of cavity wall 1057A-D. In this manner, cavity surface 1055A and protrusion surface 1065A will make contact before respective inner surfaces 1054 and 1064 make contact during a compression action. Inclusion of the aforementioned structure ensures that there is no minimum depth between surfaces 1055A, 1065A that cannot be subject to compression when frame 1010 is operated. In other variations, a depth of protrusion 1065 may be less that a depth of cavity walls 1057A-D if such minimum depth devoid of compression is desirable for an intended use.

While first and second inner members 1052, 1062 have been described specifically as part of frame 1010, it should be appreciated that such inner members 1052, 1062 may be used with a variety of other frame structures. In one variation, first and second inner members 1052, 1062 may be adapted for attachment to respective first and second blocks 12, 22 of frame 10 shown in FIGS. 1-4. In another variation, first and second inner members 1052, 1062 may be adapted for attachment to respective first and second outer members 712, 722 of frame 710 shown in FIGS. 29-35. In variations using frame 10 or frame 710, frame 10, 710 may be operated to compress soft tissue in the same way as described for frame 10. In still further variations, other frames may be used. For example, a frame may be arranged so that first inner member 1052 and second inner member 1062 are aligned with a support frame, e.g., centers of first and second inner members 1052, 1062 and actuation shaft controlling a spacing between such inner members may all pass through the same plane. In a subset of these examples, a frame may include an actuation shaft positioned to be coaxial with centers of first and second inner members 1052, 1062. In each of the above examples, an actuation shaft of the frame may include a threaded outer surface to interact with a complementary thread on part of the frame. In some variations, other means may be used as part of an actuation mechanism to control spacing between first inner member 1052 and second inner member 1062. For instance, an actuation member may include a series of notches sized to complement teeth in the frame, with a handle or other control usable to control a position of first inner member 1052 relative to second inner member 1062.

Frame 10, 710, 1010 may be made of materials that are acceptable for use in and around a surgical theater and in settings where surgical instrumentation is used in preparation for surgery. The frame may be made of polymeric materials, composites including polymeric and other materials, metals, or other materials that are easy to clean and maintain. In one specific example, first and second outer members 712, 722 and first and second inner members 732, 742, all of frame 710, are made of a clear anodized aluminum. In another example, such outer and inner members 712, 722, 732, 742 are made of stainless steel. The above-described material selections may similarly be employed for frame 10 and frame 1010 (e.g., first and second inner members 1052, 1062). Shafts, rails, supports and various other operable components may be made of metallic materials. Handles and other controls may be made of any desirable materials, and in some examples may be polymeric.

In another aspect, the present disclosure relates to a cutting guide used to prepare cuts in bone for the extraction of a bone segment, i.e., allograft, for implantation into a patient. Throughout the disclosure, the cutting guide may also be referred to simply as a guide. In some embodiments, the cutting guide is configured to attachment to a bone, such as a donor bone.

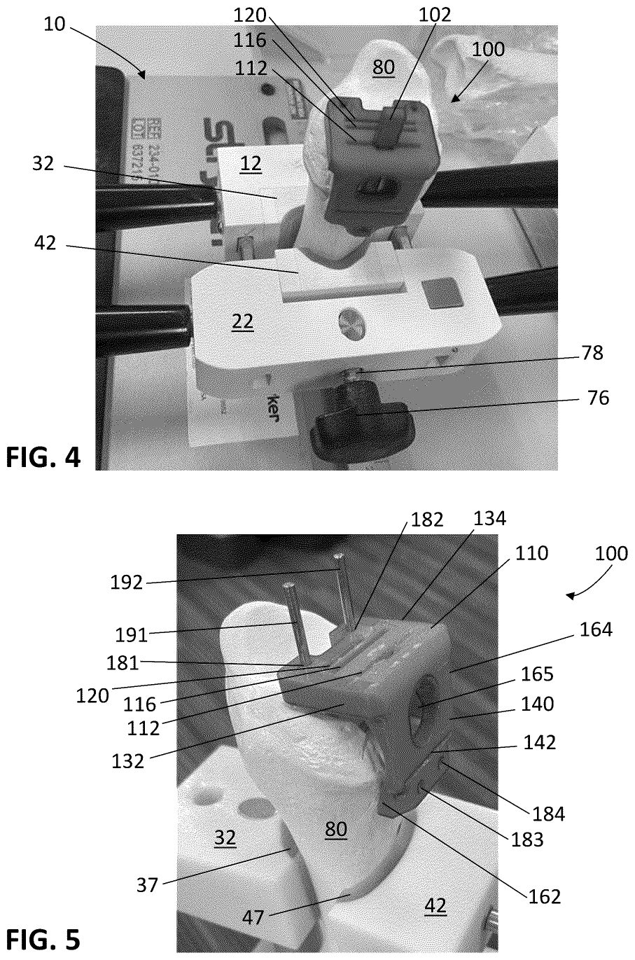

One embodiment of the cutting guide is guide 100 shown in FIGS. 4-6. As shown, guide 100 is a monolithic structure and is formed monolithically. In variations, the guide may be formed in separate parts and assembled. Such variations in formation and assembly may be utilized for all cutting guides contemplated by the present disclosure. Guide 100 includes end plate 110 and side plate 140 extending from one side of end plate 110 at an approximately ninety-degree angle relative to end plate 110. In variations, an angle between the end plate and the side plate may be greater than or less than ninety degrees.

End plate 110 has a depth that extends from an outer surface of end plate 110 to a bone-facing surface of end plate 110 opposite the outer surface and a width extending from a first side 132 to a second side 134. End plate 110 also includes a first slit 112, a second slit 116 and a third slit 120, each having a length from a first end to a second end where the first end is near but spaced apart from first side 132 and the second end is near but spaced apart from second side 134. First, second and third slits 112, 116, 120 are through slits and extend through an entirety of the depth of end plate 110. And, first, second and third slits 112, 116, 120 are parallel or close to parallel in orientation. A spacing of the respective slits may be arranged so that first slit 112 may be used as a baseline cutting location, while second and third slits 116, 120 may be alternatives for a second cut to create a cut of material that either has a dimension from the first to second slit or from the first to third slit. Second and third slits may have cutting planes that are at an angle relative to first slit such that a cut through the second or third slit and a cut through the first slit may converge in a direction moving away from the guide.

Side plate 140 has a depth that extends from an outer surface of side plate 140 to a bone-facing surface of side plate 140 opposite the outer surface and a width extending from a first side 162 to a second side 164. Side plate 140 includes side slit 142 having a length from a first end to a second end where the first end is near but spaced apart from first side 162 and the second end is near but spaced apart from second side 164. Side slit 142 is a through slit and extends through an entirety of the depth of side plate 140. Side plate 140 also includes a cylindrical opening 165 extending from an outer surface to a bone-facing surface of side plate 140. The inclusion of cylindrical opening 165 provides additional visibility of a bone surface when guide 100 is disposed thereon. Such visibility may aid in the positioning of guide 100 on the bone. In variations, opening 165 may have another shape.

Plate also includes holes 181, 182, 183, 184. In variations, the guide may have a different quantity of holes and the holes may be located at different surface locations on the guide relative to guide 100. Hole location may be dictated by space on the guide and by the anchorage expected with specific hole locations. Holes 181, 182, 183, 184 are sized to receive pins or other anchors, e.g., pins 191, 192 shown in FIG. 5, to secure the guide to an object, such as a bone. One or more of the holes may be angulated in an inward direction to optimize anchorage of anchors that are advanced through and disposed in the holes. While guide 100 is made of an opaque material as shown in FIGS. 4-6, it should be appreciated that the guide may also be made of a transparent material with transparency sufficient such that when guide 100 is positioned on a bone surface, the bone surface is visible through guide 100. Thus, when a transparent variation of guide 100 is positioned on a bone, a user can visualize a cutting path of the slots through the underlying bone surface and use such information to evaluate whether the guide should be affixed to the bone in that position or whether it should be repositioned.

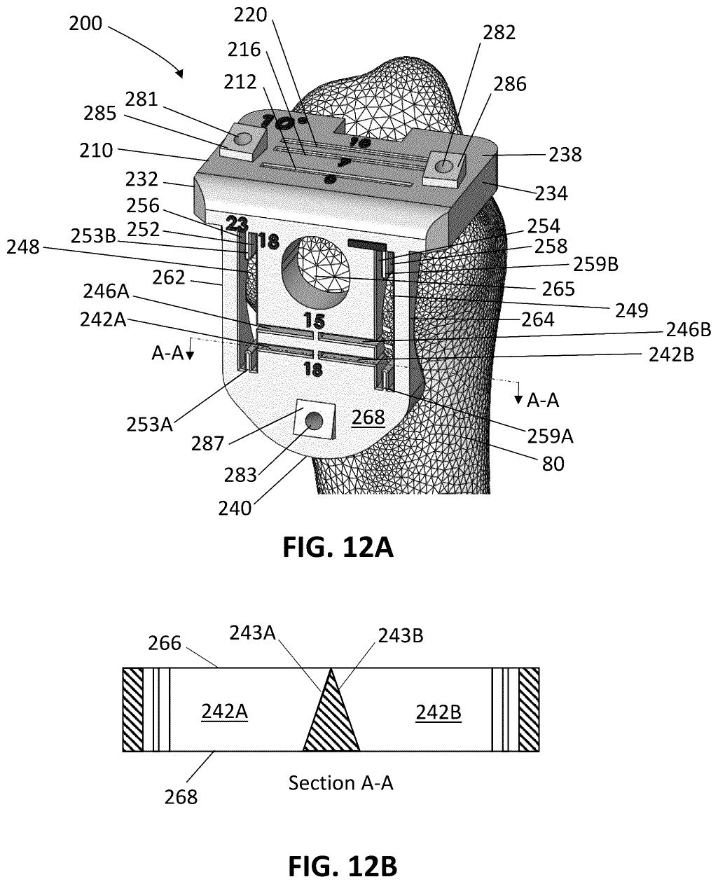

Another embodiment of cutting guide is guide 200 shown in FIGS. 12A-14. Unless otherwise indicated, like reference numerals refer to like elements of guide 100 shown in FIGS. 4-6, but within the 200-series of numerals. Guide 200 includes an end plate 210 and a side plate 240 extending from one side of end plate 210 at an approximately ninety-degree angle relative to end plate 210. In variations, an angle between the end plate and the side plate may be greater than or less than ninety degrees.

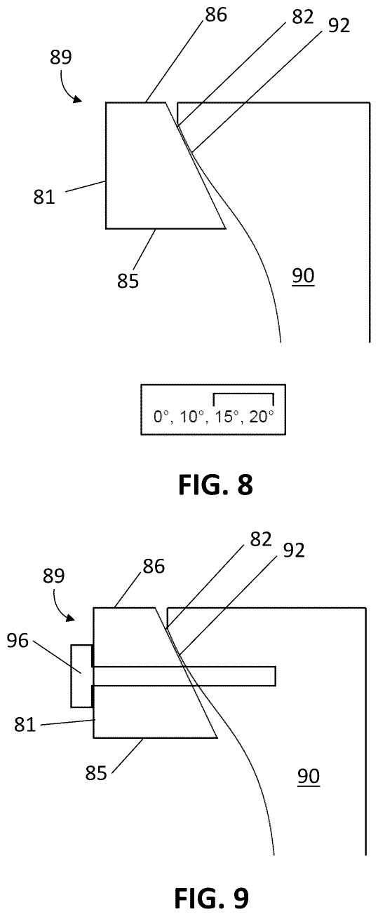

End plate 210 has a depth that extends from an outer surface 238 of end plate 210 to a bone-facing surface 236 of end plate 210 opposite the outer surface, a width extending from a first side 232 to a second side 234, and a length extending from side plate 240 to end side 231. End plate 210 also includes a first slit 212, a second slit 216 and a third slit 220, each having a length from a first end to a second end where the first end is near but spaced apart from first side 232 and the second end is near but spaced apart from second side 234. First, second and third slits 212, 216, 220 are through slits and extend through an entirety of the depth of end plate 210. And, first, second and third slits 212, 216, 220 are parallel or close to parallel in orientation at outer surface 238. However, internal cutting surfaces of the respective slits may be angled with respect to each other. For example, first slit 212 may have inner surfaces defining a cutting plane that is approximately 90 degrees relative to outer surface 238, while second and third slits 216, 220 may have respective inner surfaces defining respective cutting planes that are at an acute angle relative to outer surface 238. In FIG. 12A specifically, an angle of second and third slits 216, 220 is 10 degrees relative to first slit 212, as indicated by the label on outer surface 238. In other examples, guide 200 may be designed and fabricated so that an angle of second and third slits 216, 220 is 0 degrees, 5 degrees or 15 degrees, among other values, relative to first slit 212. A spacing of the respective slits may be arranged so that first slit 212 may be used as a baseline cutting location, while second and third slits 216, 220 may be alternatives for a second cut to create a cut of material that either has a dimension from the first to second slit or from the first to third slit. In this manner, cutting a bone via first and second slits 212, 216 or first and third slits 212, 220 may produce an allograft with a shape as shown in FIGS. 8 and 9, discussed in greater detail elsewhere in the present application.

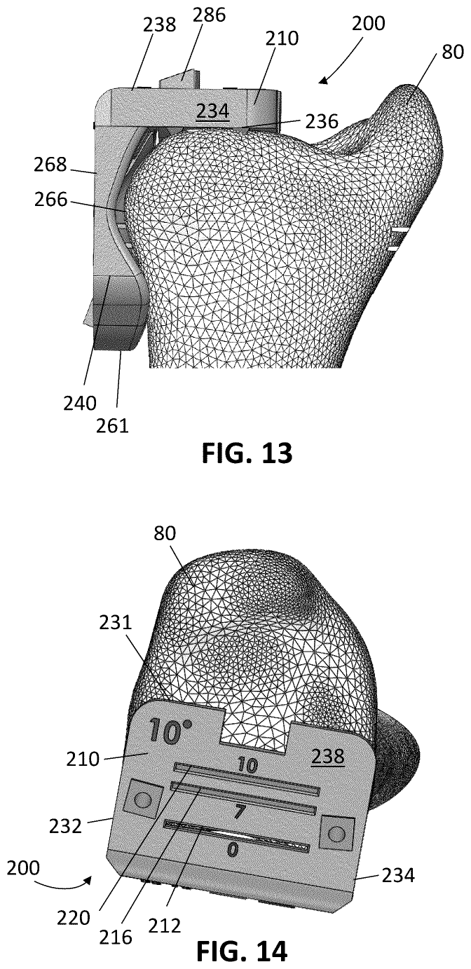

Side plate 240 has a length from a first end abutting end plate 210 to a free end 261 and a width from a first side 262 to a second side 264, as shown in FIG. 12A, and a depth from an outer surface 268 to a bone-facing surface 266, as shown in FIG. 13. Bone-facing surface 266 has a contour shaped to correspond to a bone surface to which guide 200 may be attached, such as a distal tibia. A surface contour of bone-facing surface may be varied in many ways during the design process, further details of which are described elsewhere in the present disclosure.

Side plate 240 includes several cutting slits including first elongate opening 248 and second elongate opening 249, each having a length extending along a direction orthogonal to end plate 210. Each elongate opening 248, 249 is subdivided into two slits. Specifically, first elongate opening 248 includes first inner slit 252 and first outer slit 256, and second elongate opening 249 includes second inner slit 254 and second outer slit 258. As to first elongate opening 248, respective inner and outer slits 252, 256 are separated by projections 253A-B extending inward from opposing ends of a length of first elongate opening 248, as shown in FIG. 12A. Such projections 253A-B provide structure to guide a cutting tool to cut along a plane associated with a desired slit within first elongate opening 248. Second elongate opening 249 is the same as first elongate opening 248 in the above respects, in that second inner slit 254 and second outer slit 258 are separated by projections 259A-B extending inward from opposing ends of a length of second elongate opening 249, as shown in FIG. 12A. Inner wall surfaces defining respective elongate openings 248, 249 are approximately orthogonal to outer surface 268 of side plate 240. In variations, an angle of such wall surfaces may be varied to produce a different cut angle relative to the bone being cut with guide 200.

Side plate 240 also includes a pair of upper cross-slits 246A, 246B and a pair of lower cross-slits 242A, 242B, each being oriented across a portion of the width of side plate 240 between first elongate opening 248 and second elongate opening 249. Specifically, first upper cross-slit 246A extends from elongate opening 248 to a terminal end near a centerline of side plate 240 and second upper cross-slit 246B extends from elongate opening 249 to a terminal end near the centerline of side plate 240. In this way, each upper cross-slit 246A, 246B passes through a single plane, but is separated by a small part of a body of side plate 240, as shown in FIG. 12A. First and second lower cross-slits 242A, 242B are the same as upper cross-slits 246A, 246B in the above respects, and are parallel to and spaced apart from upper cross-slits 246A, 246B. At the terminal ends of cross-slits 242A-B, 246A-B, lateral sidewalls extending through side plate 240 are tapered so that a cutting path on a bone-facing side of the guide 200 may be wider than a length of respective cross-slits 242A-B, 246A-B at outer surface 268. This is shown for cross-slits 242A-B in FIG. 12B, where lateral sidewalls 243A, 243B taper from outer surface 268 toward bone-facing surface 266, thereby providing a cutting path such that when cuts are made on both cross-slits 242A, 242B, it is possible to cut continuously across a dimension spanning both cross-slits. In other embodiments, lateral sidewalls may be at different angles than that shown in FIG. 12B, and in some instances, may simply be perpendicular to outer surface 268. As depicted in FIG. 12A, each cross-slit 242A-B, 246A-B defines a cutting plane that is orthogonal to outer surface 268. In some examples, an angulation of such cutting planes may vary and may be at an acute angle relative to outer surface 268.

Guide 200 also includes a plurality of anchor guides, with three included on guide 200 in FIGS. 12A-14. In variations, two, four or more anchor guides may be included on the guide. In other variations, the guide may simply have holes in the end plate and/or the side plate for anchorage of the guide to an object, such as bone. Turning to the details of the anchor guides, guide 200 includes first, second and third anchor guides 285, 286, 287. Each anchor guide 285, 286, 287 includes a body with a respective central hole 281, 282, 283 extending therethrough. The body of each anchor guide 285, 286, 287 includes a tapered upper surface that is perpendicular to a longitudinal axis of central hole 281, 282, 283 through the body. In this manner, when an anchoring member such as a pin is passed into the hole, the guide steers the pin along the predetermined angle of the hole. In the depicted arrangement, first anchor guide 285 is positioned on end surface 238 adjacent to first side 232, second anchor guide 286 is positioned on end surface 238 adjacent to second side 234 and third anchor guide 287 is positioned on side surface adjacent to free end 261.

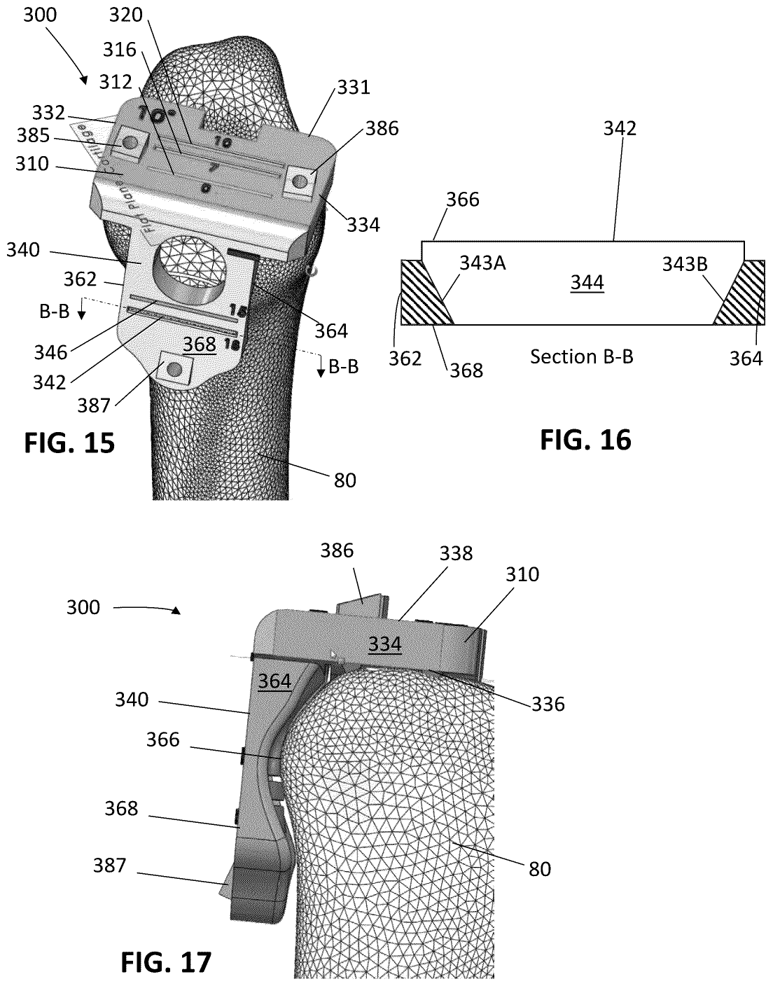

Another embodiment of cutting guide is guide 300 shown in FIGS. 15-17. Unless otherwise indicated, like reference numerals refer to like elements of guide 100 shown in FIGS. 4-6, but within the 300-series of numerals. Guide 300 includes an end plate 310 and a side plate 340 extending from one side of end plate 310 at an approximately ninety-degree angle relative to end plate 310. In variations, an angle between the end plate and the side plate may be greater than or less than ninety degrees.

End plate 310 has a depth that extends from an outer surface 338 of end plate 310 to a bone-facing surface 336 of end plate 310 opposite the outer surface and a width extending from a first side 332 to a second side 334. End plate 310 also includes a first slit 312, a second slit 316 and a third slit 320, each having a length from a first end to a second end where the first end is near but spaced apart from first side 332 and the second end is near but spaced apart from second side 334. First, second and third slits 312, 316, 320 are through slits and extend through an entirety of the depth of end plate 310. And, first, second and third slits 312, 316, 320 are parallel or close to parallel in orientation at outer surface 338. However, internal cutting surfaces of the respective slits may be angled with respect to each other. For example, first slit 312 may have inner surfaces defining a cutting plane that is approximately 90 degrees relative to outer surface 338, while second and third slits 316, 320 may have respective inner surfaces defining respective cutting planes that are at an acute angle relative to outer surface 338. A spacing of the respective slits may be arranged so that first slit 312 may be used as a baseline cutting location, while second and third slits 316, 320 may be alternatives for a second cut to create a cut of material that either has a dimension from the first to second slit or from the first to third slit.

Side plate 340 has a depth that extends from an outer surface 368 to a bone-facing surface 366 opposite outer surface 368 and a width extending from a first side 362 to a second side 364 opposite first side 362. First and second sides 362, 364 are outer side surfaces of side plate 340 and as depicted have flat, planar surfaces such that cutting may be performed using such outer side surfaces as a guide. Side plate 340 includes lower cross-slit 342 and upper cross-slit 346, each having a length from a first end to a second end where the first end is near but spaced apart from first side 362 and the second end is near but spaced apart from second side 364. Lower and upper cross-slits 342, 346 are through slits and extend through an entirety of the depth of side plate 340. And, lower cross-slit 342 and upper cross-slit 346 are parallel or close to parallel in orientation at outer surface 368. While each cross-slit 342, 346 is spaced from respective first and second sides 362, 364 at outer surface 368, internal sidewalls of each slot taper outward such that the slits become wider toward bone-facing surface 366. This is shown through a top-down cross-sectional view of side plate 340 in FIG. 16. Specifically, lower cross-slit 342 has a first lateral sidewall 343A and a second lateral sidewall 343B defining a width of lower cross-slit 342, with the respective lateral sidewalls 343A-B moving away from each other toward bone-facing surface 366. A planar cutting surface 344 of lower cross-slit 342 is also shown in FIG. 16. Upper cross-slit 346 has a similar shape as lower cross-slit 342. In variations, the depicted taper may also be understood to be a bevel within the sides of the slit. Both the outer side surfaces of first and second sides 362, 364 and surfaces defining at least part of lower and upper cross-slits 342, 346 are guide surfaces adapted to facilitate cutting along a predetermined path on side plate 340. As with guide 200, bone-facing surface 366 of side plate 340 is contoured to conform to a bone surface to which it is pressed against during use, as shown in FIG. 17. Guide 300 also includes a plurality of anchor guides 385, 386, 387 in the same matter as provided in guide 200.

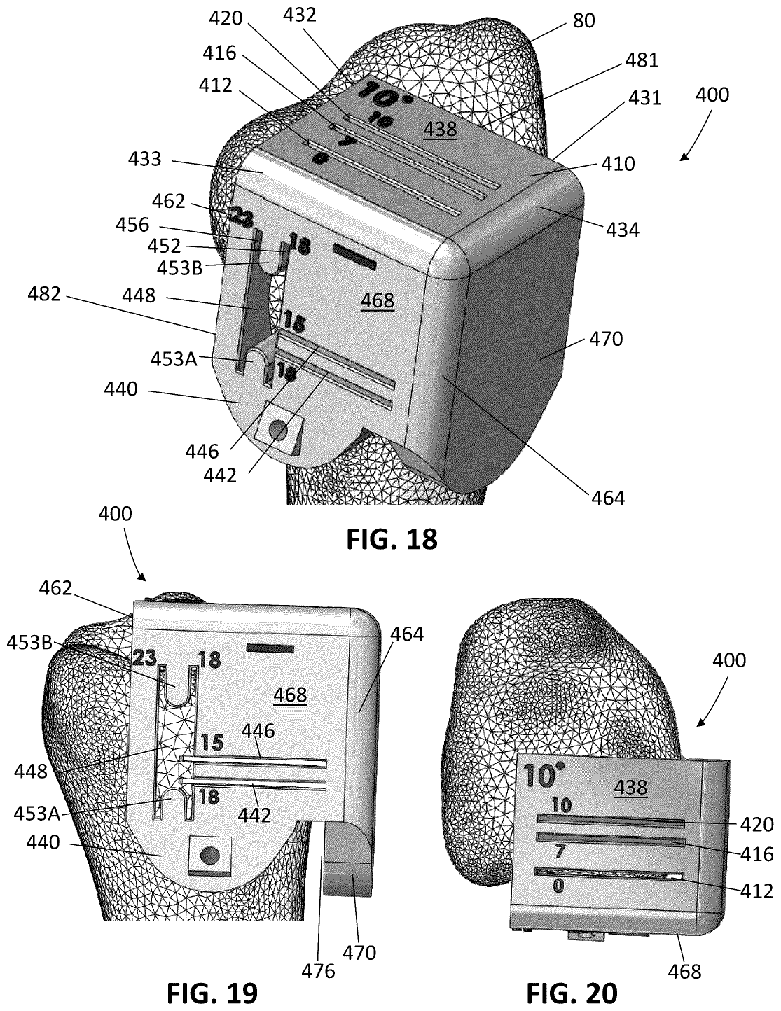

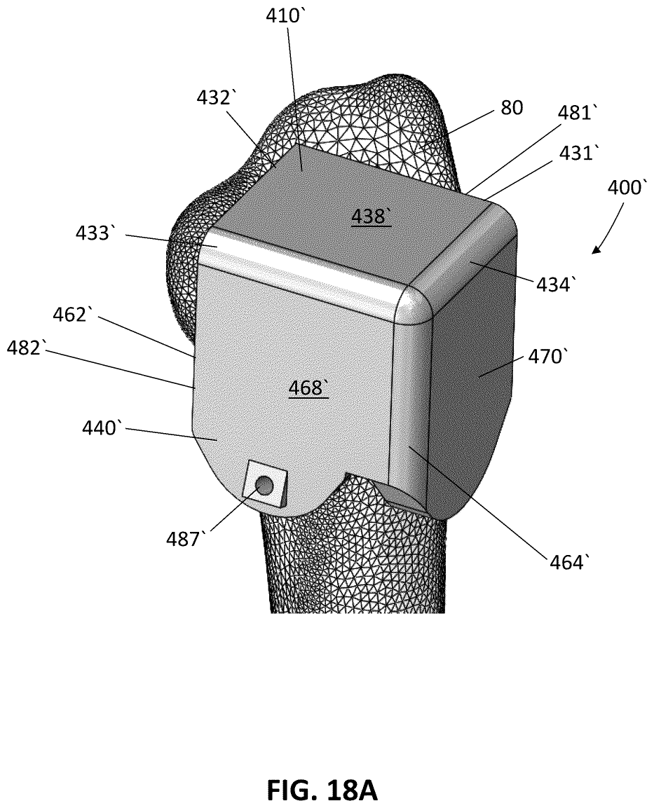

Another embodiment of cutting guide is guide 400 shown in FIGS. 18-20. Unless otherwise indicated, like reference numerals refer to like elements of guide 100 shown in FIGS. 4-6, but within the 400-series of numerals. Guide 400 includes an end plate 410 and a first side plate 440 extending from one side of end plate 410 at an approximately ninety-degree angle relative to end plate 410 and a second side plate 470 extending from a second side of end plate 410 at an approximately ninety-degree angle relative to end plate 410. First side plate 440 and second side plate 470 are also joined directly, separate from end plate 410 as shown in FIG. 18. In variations, an angle between end plate 410 and one or both of the first side plate 440 and the second side plate 470 may be greater than or less than ninety degrees.

End plate 410 has a depth that extends from an outer surface 438 of end plate 410 to a bone-facing surface (not shown) of end plate 410 opposite the outer surface, a width extending from a first side 432 to a first transition region 434 and a length from a second side 431 to a second transition region 433. On second side 431 of end plate 410 transverse to outer surface 438 is an outer side surface 481 of end plate 410. Outer side surface 481 may be used as a guide surface for a cutting tool. An outer side surface on first side 432 may also serve the same function. End plate 410 also includes a first slit 412, a second slit 416 and a third slit 420, each having a length from a first end to a second end where the first end is near but spaced apart from first side 432 and the second end is near but spaced apart from first transition region 434. First, second and third slits 412, 416, 420 are through slits and extend through an entirety of the depth of end plate 410. And, first, second and third slits 412, 416, 420 are parallel or close to parallel in orientation at outer surface 438. However, internal cutting surfaces of the respective slits may be angled with respect to each other. For example, first slit 412 may have inner surfaces defining a cutting plane that is approximately 90 degrees relative to outer surface 438, while second and third slits 416, 420 may have respective inner surfaces defining respective cutting planes that are at an acute angle relative to outer surface 438. A spacing of the respective slits may be arranged so that first slit 412 may be used as a baseline cutting location, while second and third slits 416, 420 may be alternatives for a second cut to create a cut of material that either has a dimension from the first to second slit or from the first to third slit.

First side plate 440 has a depth that extends from outer surface 468 to a bone-facing surface (not shown) opposite outer surface 468 and a width extending from a first side 462 to a third transition region 464 opposite first side 462. On first side 462 of first side plate 440 transverse to outer surface 468 is an outer side surface 482 of first side plate 440. Outer side surface 482 may be used as a guide surface for a cutting tool. First side plate 440 includes elongate opening 448 having a length extending along a direction orthogonal to end plate 410. Elongate opening 448 is subdivided into two slits, inner slit 452 and outer slit 456. Inner and outer slits 452, 456 are separated at opposite ends of elongate opening 448 by lower projection divider 453A and upper projection divider 453B, respectively, as shown in FIG. 18. Each projection divider 453A-B in the depicted embodiment includes a rounded free end, although it is contemplated that the shape of the projection divider may vary from that shown. First side plate 440 also includes lower cross-slit 442 and upper cross-slit 446, each having a length from a first end that opens into elongate opening 448 to a second end near but spaced apart from third transition region 464. Lower and upper cross-slits 442, 446 are through slits and extend through an entirety of the depth of side plate 440. And, lower cross-slit 442 and upper cross-slit 446 are parallel or close to parallel in orientation at outer surface 468. The bone-facing surface of first side plate 440 may be contoured in the manner shown for guides 200, 300, or it may be designed to have a contour to generally correspond with or match bone surfaces other than that shown in the Figures, as is also contemplated for other guides of the present disclosure.

Second side plate 470 extends from end plate 410 and first side plate 440 in a continuous manner such that the overall guide 400 forms a solid enclosed structure among these three parts. As depicted, second side plate 470 is solid and includes a bone-facing surface 476. Bone-facing surface 476 of second side plate 470 may be contoured in the manner shown for side plate 240, 340 in respective guides 200, 300, or it may be designed to have a contour to generally correspond with or match bone surfaces other than that shown in the Figures. For both side plates, designs may be varied so that bone-facing surfaces may have contours other than those explicitly designed to complement bone surfaces.

Another embodiment of cutting guide is guide 400′ shown in FIG. 18A. Unless otherwise indicated, like reference numerals refer to like elements of guide 400 shown in FIGS. 18-20, but within the 400-series of numerals. Guide 400 includes an end plate 410′ and first and second side plates 440, 470′ that are the same as those included in guide 400. The plates of guide 400′ may be generally solid structures without cutting slits, as shown in FIG. 18A. Guide 400′ may include one or more openings to receive anchors usable to secure guide 400 to bone. While FIG. 18A illustrates only a single anchor guide 487 to receive an anchor, it should be appreciated that two or more anchor guides may be used and that such anchor guides may be located at any desired plate location on guide 400. Outer side surface 481′ of end plate 410′ may be used as a guide surface for a cutting tool and similarly outer side surface 482′ of first side plate 440′ may also be used as a guide surface for a cutting tool.

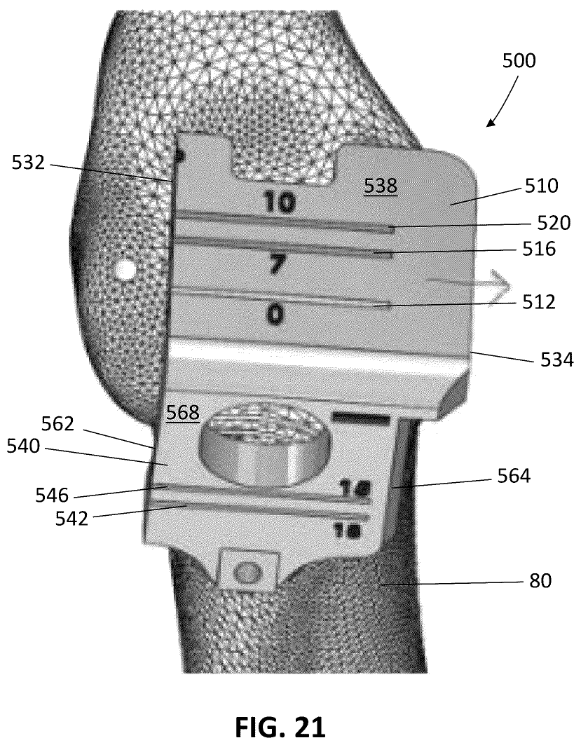

Another embodiment of cutting guide is guide 500 shown in FIG. 21. Unless otherwise indicated, like reference numerals refer to like elements of guide 100 shown in FIGS. 4-6, but within the 500-series of numerals. As with guide 400, guide 500 includes an end plate 510 with an outer surface 538 and first, second and third slits 512, 516, 520, and a side plate 540 with an outer surface 568 and lower and upper cross-slits 542, 546. A difference in guide 500 is that first sides 532, 562, are finished to define a cutting plane, with each slit 512, 516, 520, 542, 546 extending directly into first sides 532, 562, as shown. Although not visible in FIG. 21, guide 500 may also include a second side plate positioned relative to a remainder of the guide in a similar manner as second side plate 470 included in guide 400.

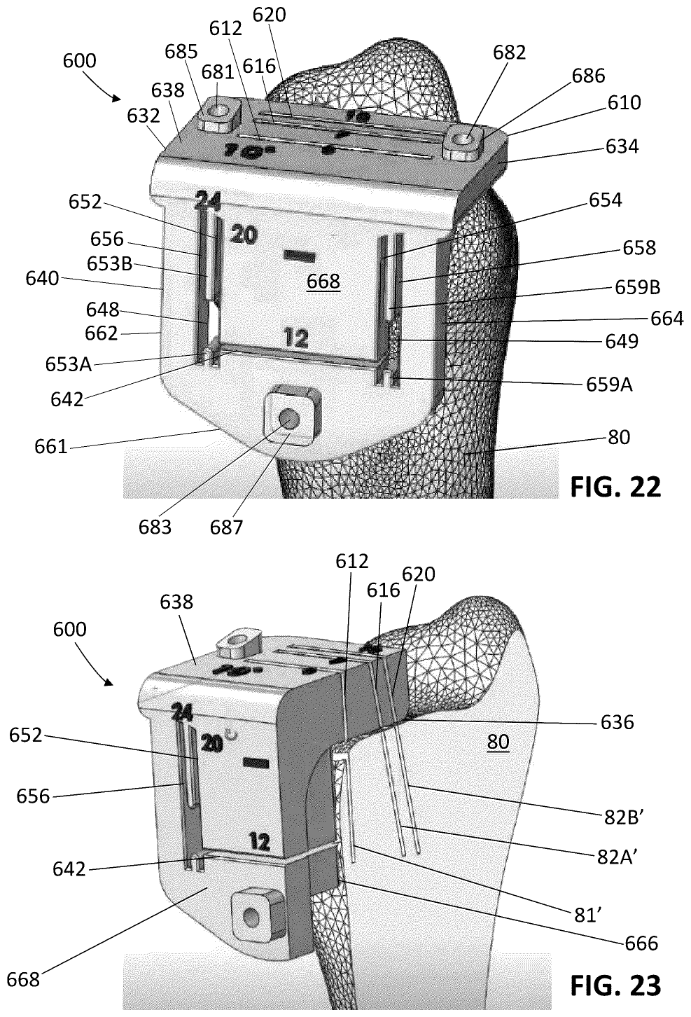

Another embodiment of cutting guide is guide 600 shown in FIGS. 22-28. Unless otherwise indicated, like reference numerals refer to like elements of guide 200 shown in FIGS. 12-14, but within the 600-series of numerals. Guide 600 includes an end plate 610 and a side plate 640 extending from one side of end plate 610 at an approximately ninety-degree angle relative to end plate 610. In variations, an angle between the end plate and the side plate may be greater than or less than ninety degrees.

End plate 610 has a depth that extends from an outer surface 638 of end plate 610 to a bone-facing surface 636 of end plate 610 opposite the outer surface and a width extending from a first side 632 to a second side 634. End plate 610 also includes a first slit 612, a second slit 616 and a third slit 620, each having a length from a first end to a second end where the first end is near but spaced apart from first side 632 and the second end is near but spaced apart from second side 634. A length of each of first, second and third slits 612, 616, 620 may correspond to a distance between first and second outer slits 656, 658 on side plate 640, described in greater detail below. In the depicted example, such distance is 24 mm. First, second and third slits 612, 616, 620 are through slits and extend through an entirety of the depth of end plate 610. And, first, second and third slits 612, 616, 620 are parallel or close to parallel in orientation at outer surface 638. However, internal cutting surfaces of the respective slits are angled with respect to each other, as shown in FIG. 23. Specifically, first slit 612 may have inner surfaces defining a cutting plane that is approximately 90 degrees relative to outer surface 638, while second and third slits 616, 620 have respective inner surfaces defining respective cutting planes that are at a 10 degree angle relative to first slit 612, as indicated by the label on outer surface 638. A cut through the bone made through these slits is also illustrated in FIG. 23, where a first planar bone surface 81′ is created by a cut through first slit 612, a second planar bone surface 82A′ is created by a cut through second slit 616 and a third planar bone surface 82B′ is created by a cut through third slit 620. In other examples, guide 600 may be designed and fabricated so that an angle of inner surfaces that define second and third slits 616, 620 is 0 degrees, 5 degrees or 15 degrees, among other values, relative to inner surfaces that define first slit 612. In still further examples, first slit 612 may be at a non-perpendicular angle relative to outer surface 638. For instance, opposing inner surfaces defining first slit may be 5, 10 or 15 degrees relative to a plane normal to outer surface 638, where such inner surfaces of first slit 612 move further away from side plate 640 toward a bone-facing surface of end plate 610. In these examples, an angle of second slit 616 and third slit 620 may be parallel to that of first slit 612 or may be non-parallel. A spacing of the respective slits may be arranged so that first slit 612 may be used as a baseline cutting location, while second and third slits 616, 620 may be alternatives for a second cut to create a cut of material that either has a dimension from the first to second slit or from the first to third slit. In this manner, cutting a bone via first and second slits 612, 616 or first and third slits 612, 620 may produce an allograft with a shape as shown in FIGS. 8 and 9, discussed in greater detail elsewhere in the present disclosure. For example, a cut through first slit 612 and second slit 616 produces a graft with a tapered shape in a direction of those cuts.

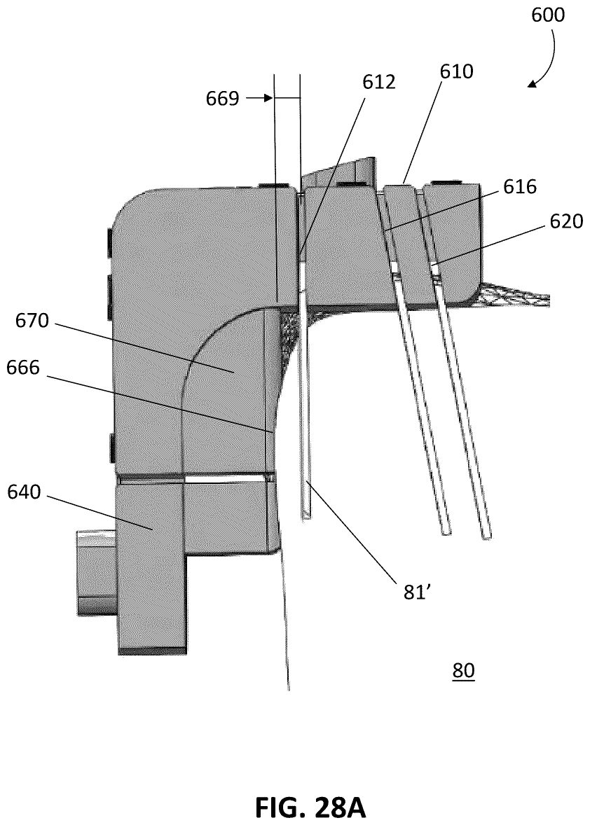

Side plate 640 has a length from a first end abutting end plate 610 to a free end 661 and a width from a first side 662 to a second side 664, as shown in FIG. 22, and a depth from an outer surface 668 to a bone-facing surface 667, as shown in FIGS. 25 and 28.

Side plate 640 includes several cutting slits including first elongate opening 648 and second elongate opening 649, each having a length extending along a direction orthogonal to end plate 610. Each elongate opening 648, 649 is subdivided into two slits. Specifically, first elongate opening 648 includes first inner slit 652 and first outer slit 656, and second elongate opening 649 includes second inner slit 654 and second outer slit 658. As to first elongate opening 648, first inner slit 652 and first outer slit 656 are separated by projections 653A-B extending inward from opposing ends of a length of first elongate opening 648, as shown in FIG. 22. Such projections 653A-B provide structure to guide a cutting tool to cut along a plane associated with a desired slit within first elongate opening 648. Second elongate opening 649 is the same as first elongate opening 648 in the above respects, in that second inner slit 654 and second outer slit 658 are separated by projections 659A-B extending inward from opposing ends of a length of second elongate opening 649, as shown in FIG. 22. Inner wall surfaces defining respective first and second elongate openings 648, 649 are approximately orthogonal to outer surface 668 of side plate 640. In use, an allograft with a 20 mm width may be extracted from a donor bone through cuts made through first inner slit 652 and second inner slit 654 and an allograft with a 24 mm width may be extracted from a donor bone through cuts made through first and second outer slits 656 and 658. In the depicted embodiment, and as indicated above, end plate 610 slits may be dimensioned to match a distance between respective first and second outer slits 656, 658. Thus, when first and second outer slits 656, 658 are 24 mm apart, a length of first, second and third slits 612, 616, 620 may also be 24 mm. In variations, an angle of such wall surfaces may be varied to produce a different cut angle relative to the bone being cut with guide 600.

Side plate 640 also includes a cross-slit 642 oriented across a portion of the width of side plate 640 between first elongate opening 648 and second elongate opening 649 such that cross-slit 642 extends into first elongate opening 648 at a first end and into second elongate opening 649 at a second end opposite the first end. Put another way, there is no physical obstruction between cross-slit 642 and first and second elongate openings 648, 649. On outer surface 668 and/or through a thickness of side plate 640, a length direction of cross-slit 642 may be orthogonal to a length direction of first and second elongate openings 648, 649. Internal surfaces of cross-slit 642 are oriented perpendicular to outer surface 668 and the position of cross-slit 642 on the side plate 640 is such that a cut through cross-slit 642 when the guide is anchored to a bone produces a bone extraction with a vertical dimension of 12 mm. The angulation of the slit and its location on the slit may be varied to suit other representative designs.

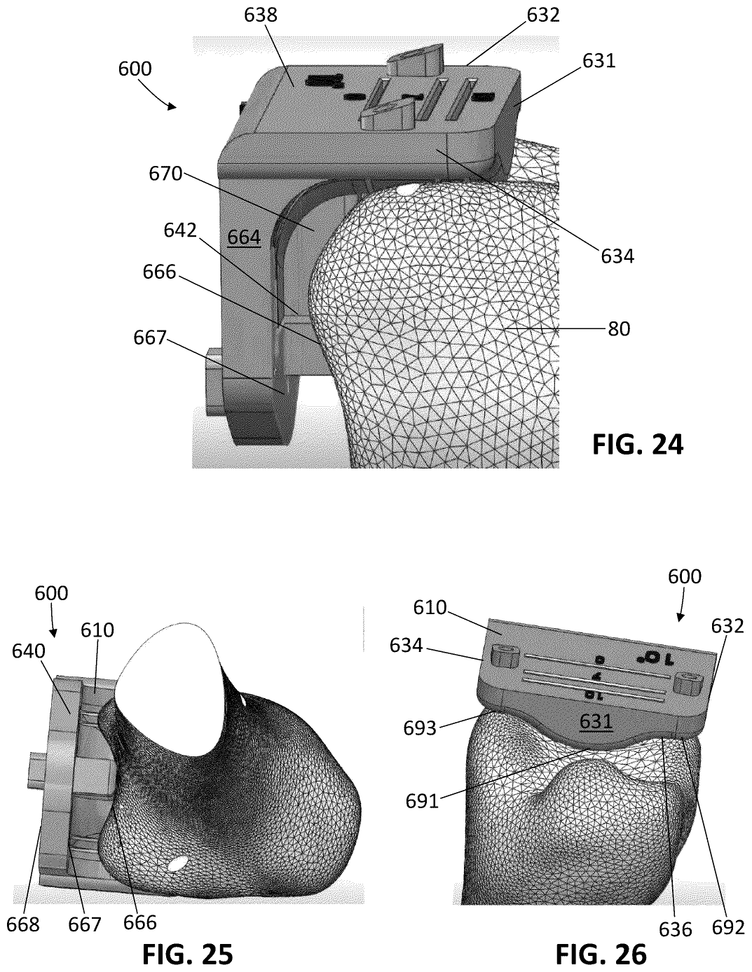

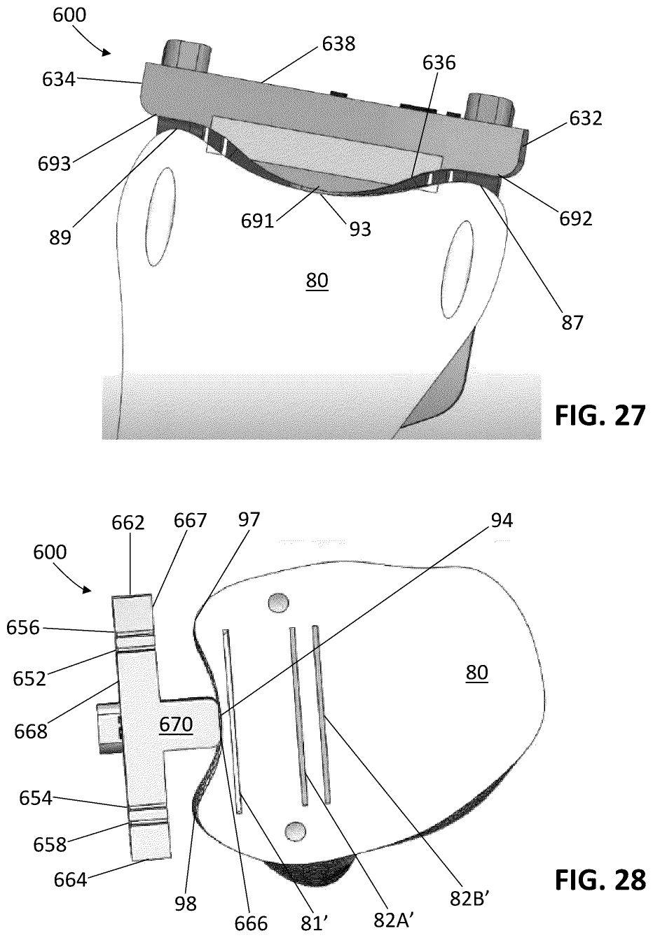

Further to the above-described features of end plate 610 and side plate 640, guide 600 also includes an anatomically shaped bone-facing surface 636 on end plate 610, as shown in FIGS. 26-27, and a spacer 670 extending from bone-facing surface 667 of side plate 640, as shown in FIGS. 24-25 and 28. Beginning with end plate 610, bone-facing surface 636 includes a protruding central region sized and positioned to fit between ridges of a distal tibia or other bone to which the guide will be attached. With a view to FIG. 27, bone-facing surface 636 includes generally flat first and second peripheral surfaces 692, 693 on opposite sides of the bone-facing surface, with those surfaces curving away from outer surface 638 moving toward a center of end plate 610 until reaching a convex apex 691, such that a maximum thickness of end plate 610 is at convex apex 691. A region of end plate 610 encompassing convex apex 691 may also be referred to as an end plate central protrusion. This design allows convex apex 691 of bone-facing surface 636, extending from end 631 toward side plate 640, to fit within, i.e., be positionable on end trough 93 of distal tibia 80. The shape of bone-facing surface 636 and an overall thickness of end plate 610 is designed using an aggregation of data from a collection of bones, described in greater detail elsewhere in the present disclosure, to optimize the dimensions so that guide 600 may be employed on a wide variety of donor bone shapes. In this manner, even if a size, shape and/or location of end trough 93, first end ridge 87 or second end ridge 89 of distal tibia 80 varies from that shown in FIG. 27, guide 600 will still be securable onto end trough 93 to obtain a reliable engagement and alignment with the bone. As seen in FIG. 27, a gap between first peripheral surface 692 and first end ridge 87 provides room to fit the guide onto a bone even when the bone is larger or has a steeper end ridges.

As to side plate 640, spacer 670 extends outward from bone-facing surface 667 as shown in FIGS. 24, 25, 28 and 28A. While side plate 640 has a generally constant thickness, spacer 670 projects from side plate 640 and includes a bone-facing surface 666. A dimension of spacer 670 from bone-facing surface 667 to bone-facing surface 666 and its position along a width of side plate 640 between first side 662 and second side 664 may be established through an analysis of an aggregation of data for distal tibia bones so that the design best fits a side trough 94 on the distal tibia, as shown in FIG. 28, while also simultaneously fitting under end plate 610 as described above, and so that side plate 640 is oriented to have cut lines through inner slits 652, 654 and outer slits 656, 658 that are perpendicular to first slit 612. A design that realizes this function may have outer surface 668 of side plate 640 oriented at a small angle relative to an axis passing through both first side facing ridge 97 and second side facing ridge 98, as shown in FIG. 28. With the inclusion of spacer 670, the realization of such an arrangement is simplified because side plate 640 is spaced apart from a bone surface even when guide 600 is in contact with distal tibia 80 for anchorage of guide 600. Through the use of data aggregation, design outputs may be such that an axis along a length of convex apex 691 may be offset from a central plane through a height of spacer 670 (the height having a height dimension) that also passes through side plate 640. Such offset, where present, results from the offset between natural locations of end trough 93 and side trough 94 on the distal tibia 80 that is generated through data aggregation or that is present in any individual specimen. Moreover, spacer 670 and first, second and third slits 612, 616, 620 on end plate 610 may be sized and positioned to optimize a location of first slit 612 relative to bone-facing surface 666 of spacer 670. Specifically, and as shown in FIG. 28A, a plane through first slit 612, which, when utilized for a bone cut, forms a cut through the bone to define first planar bone surface 81′, is generally parallel to and offset from bone-facing surface 666 by an offset distance 669. Guide 600 is designed so that offset distance 669, when guide 600 is pressed against bone such as is shown in FIG. 28A, produces a cut through distal tibia 80 such that only a small amount of curvature on an end surface of distal tibia 80 is captured between the cut through first slit 612 and the cut through second or third slit 616, 620. In this way, the cut through first slit 612 avoids merely scraping the side surface of the bone or otherwise leaving part of a cortical bone surface on a side of the bone intact. And, while spaced apart from the side face, the cut location is also close enough to the side face of the bone so that a small amount of the curvature on the end surface of distal tibia 80 is captured between the end surface cuts. Retrieving an allograft based on retrieval at a depth established by slits in guide 600 produces an allograft found to be optimal relative to an allograft based on a first baseline endplate cut closer to a side of the bone or deeper into the bone relative to the arrangement shown in FIG. 28A.

Guide 600 also includes three anchor guides 685, 686, 687 for use in anchoring guide 600 to bone. These anchor guides may be varied in position and number as described elsewhere in the present disclosure. While guide 600 may have applicability in bones other than a distal tibia, in other embodiments, a guide may be designed with inner surfaces tailored for other bone types, such as a distal radius. In such cases, guides for other bone types may be designed using data aggregation techniques as described above for guide 600.

The guides described above and otherwise contemplated by the present disclosure may be made of a variety of biocompatible materials. For example, the guides may be made of polymeric materials. Polymeric materials used may be opaque or transparent so that objects such as bone are viewable through the guide. In other examples, the guide may be made of a metallic material. Examples of metallic materials that may be used to form the guide include stainless steel and titanium. In some examples where metallic materials are used, such metallic materials may be treated prior to or during the fabrication process.

The guide described herein may be varied in many ways. Each of the guides contemplated herein may be formed monolithically. In all contemplated guide embodiments, including those illustrated in the Figures, the guide may include one or more holes to receive an anchor, and one or more of those holes may be included within an anchor guide disposed on the guide. Additionally, the slit and plate features of any one of the described guides may be interchangeable with other slit and plate features described for other guides.

In another aspect, the present disclosure relates to a system that includes any frame contemplated by the present disclosure, such as frame 10 or frame 710, in combination with any guide contemplated by the present disclosure, such as one of guides 100, 200, 300, 400, 500, 600.

In another aspect, the present disclosure relates to a kit including two or more components for use in retrieval of an allograft and preparation of soft tissue for implantation in a patient.

In some embodiments, a kit may include a frame, one or more pairs of internal blocks receivable in the frame and two or more cutting guides. A pair of internal blocks in the kit may be first and second internal blocks 32, 42 or third and fourth internal blocks 52, 62. In other examples, the kit may include two pairs of internal blocks inclusive of internal blocks 32, 42, 52, 62. In still further examples, the kit may include additional or alternative internal blocks. In some variations that include alternative blocks, the internal blocks may be different from first and second internal blocks 32, 42 in that the internal blocks may be adapted for engagement with different types or sizes of bone. Other internal blocks may also be included as contemplated by the present disclosure. In some embodiments including some of the above-described embodiments, the internal blocks of the kit may be accompanied by inserts, and two or more pairs of inserts may be included for a single pair of internal blocks. In these embodiments, the pairs of inserts may be different from each other to complement different bone sizes and shapes, and may be removably attached to the internal blocks.

In some variations of the above embodiments, the kit may include two or more cutting guides such as two of guides 100, 200, 300, 400, 500, or another guide as contemplated by the present disclosure. Non-depicted guides that may be included in the kit include those with different cutting planes or different dimensions between slits compared to the depicted guides. Among the included guides, there may be two or more guides that are the same, two or more guides that are different from each other, or both. The guides included in these variations may include any number of guide configurations as contemplated by the present disclosure.

In some embodiments, a kit may include a frame and two or more pairs of internal blocks receivable in the frame, each pair being adapted for a different bone size or type. In some examples, one pair of internal blocks included within the kit may be first and second internal blocks 32, 42 or third and fourth internal blocks 52, 62. In some examples, the kit may include additional or alternative internal blocks. Such internal blocks may be different from first and second internal blocks 32, 42, in that those blocks may be adapted for engagement of different types or sizes of bone. Other internal blocks may also be included as contemplated by the present disclosure.

In some embodiments, a kit may include two or more cutting guides. In some examples, two or more guides may be included from among guides 100, 200, 300, 400, 500, or another guide as contemplated by the present disclosure. Non-depicted guides that may be included in the kit include those with different cutting planes or different dimensions between slits compared to the depicted guides. For example, cutting guides that may be included in the kit include those with slits on end plate, e.g., end plate 210, where first slit 212 is orthogonal to outer surface 238 and second and third slits 216, 220, are parallel, i.e., at 0 degrees relative to first slit 212, or at 5, 10 or 15 degrees relative to first slit 212. Thus, in one specific example of a kit, the kit may include four guides, where the respective end plates of the guides have sets of slits such that a second slit is 0, 5, 10 and 15 degrees relative to a first slit. In this way, the kit provides many available options for selection of an optimal cut for retrieval of an allograft. Among the included guides, there may be two or more guides that are the same, two or more guides that are different from each other, or both. The guides included in these variations may include any number of guide configurations as contemplated by the present disclosure.

In any of the above contemplated kit embodiments, the kit may further include one or more anchoring pins for use in securing a cutting guide to a bone. Further, any of the above contemplated embodiments may include implantable and biocompatible bone anchors usable to secure an allograft to a bone of a patient. Further, in any one of the above embodiments, the kit or individual items and combinations thereof may be disposed within a package or a plurality of packages. For example, all of the items of the kit may be disposed within a single package. In another example, all of the cutting guides may be in one package and all of the internal blocks in another. The items included in the kit may also be individually packaged. For example, each cutting guide may be in its own package. Packaging each item in the kit separately or in different combinations may improve the sterility of the items in preparation for use with implantable materials or in the surgical theater. In any of the above embodiments, a kit may further include an instruction manual with an explanation of details relating to the contents of the kit including instructions for use of the contents.

Another aspect of the present disclosure relates to methods of designing one or more sets of inserts for the internal blocks received in the frame, such as first and second inserts 37, 47, one or more cutting guides, such as guide 200, or both inserts and cutting guides, where such designs may be used to fabricate the respective instrumentation components for use in surgery and additionally or alternatively may be incorporated into software to assist in pre-surgical planning. We begin with the process of design itself, followed by a description of how established designs may be incorporated into user-interfacing software for pre-surgical planning purposes.

In some embodiments, a process of designing a pair of inserts and/or cutting guides begins with establishment of a reference geometry of a bone that will be engaged by the pair of inserts and/or cutting guide. The bone type that will be the basis for the design may be a tibia, radius or iliac crest, among others, although designs based on the tibia are referenced below for the sake of brevity. To establish the reference geometry, morphological bone data of a tibia, also referred to as bone data, is collected from images across an assortment of demographic profiles and is fed into a database. Such database information is then paired with software so that target bone data for a specific design is retrievable from within a substantial database of reference data located in the database. The bone data retrieved from the database for any given design may include or otherwise be based on a large number of individual CT scans. It should be appreciated that any number of data aggregation systems and techniques may be used.

Once retrieved, the bone data is analyzed using statistical modeling or other similar techniques to establish a single representative universal design or two or more representative universal designs. In one example of this process, a design may be based on representative anatomic points that are established by aggregating those points from the retrieved bone data. As is likely to be the case in many instances, where there is a wide range of values for a particular characteristic such as size among the retrieved bone data, the bone data may be divided into several groups so that each group of data may be analyzed independently to establish a universal design for that group. Thus, with a distal tibia, for example, if the bone data is divided into small, medium and large distal tibias, then each of those may be separately analyzed as a standalone data set to establish a universal design for that size. Again, this may be done so that an established tibia geometry may be used for a design of a pair of inserts to be attached to internal blocks or for a design of a cutting guide, as representative examples. Thus, in the above example based on having three sizes, a design output may be three sizes of insert pairs or three sizes of cutting guides. For cutting guides in particular, differences between sizes may be in overall dimensions, bone-facing surface contours, and/or slit size, spacing, and/or angulation through the respective plates of the cutting guide. While one differentiator between groups of bone data may be size, it should be appreciated that characteristics other than size may also be used to establish different insert and cutting guide designs. Ultimately, a pair of inserts designed using the above-described process have surface contours that are best fit to a representative tibia established through the use of aggregated bone data in a database, while cutting guides designed using the above-described process have one or more of a size, bone-facing surfaces, and/or slits that are a best fit for a representative tibia established through the use of the database.

As an extension of the above method, the designs established through the use of aggregated bone data in a database may be uploaded into software for use in pre-operative planning. Specifically, once universal pairs of inserts, cutting guides, and/or other instrumentation are designed and designated as designs that will be available for use in a surgical procedure, i.e., will be manufactured and available for procurement by a user, such designs, including the various sizes thereof, are uploaded into the software. Then, in the pre-operative planning process, a user may upload an image, e.g., CT scan of a patient requiring shoulder surgery. An image of the existing condition of the patient may then be viewed on a user interface, and the user may view a loss of glenoid bone in the patient. The software may provide a drop-down menu or other selection option so that a user may select an allograft from among those available for use as a shoulder implant in surgery. For example, there may be small, medium and large allografts as available options. These may be selected and overlaid on the image of the patient to evaluate suitability. It should be appreciated that the allograft sizes generated by the software would be produced by cutting through the slits in a complementary universal cutting guide that corresponds to an allograft selected by the user. This process simplifies the cutting guide selection process for a user. Thus, once the user finds and selects an allograft of a suitable size using the software, a cutting guide that is the same size is procured for the user.

Returning to other design approaches, in another embodiment, a cutting guide may be designed using a partial or fully patient specific approach. In some embodiments, a cutting guide may include a plurality of slits that establish cut lines where the slit size, angulation, and spacing are determined in a patient specific manner. In one example, this process may proceed by evaluating a shoulder of the patient that will receive an allograft implant as part of a surgical procedure. Through such evaluation, dimensions of the allograft to be used may be determined, along with an angulation of a surface of the allograft that will abut a bone surface of the patient to which the allograft will be anchored. One example of the anatomy being evaluated in such case is shown in FIG. 8. With such information in hand, a cutting guide may be designed and fabricated with cutting slots located and angled to produce an allograft with the sought after shape and size. In other embodiments, patient specific design techniques may be employed more broadly to design a cutting guide body or inserts for internal blocks used with the frame. In these cases, aspects of the design that relate to a bone-facing surface of a body of the cutting guide or bone-facing surfaces of the inserts are based on a surface of a specific source bone, i.e., donor bone, used to extract an allograft, and in this way are not related to a patient anatomy. In many cases, the source bone may be from a cadaver, and in this way, such design approaches require scans and/or other detailed information about the source bone in order to employ patient specific techniques to the design of the cutting guide and inserts. Nonetheless, this is an optional approach to the design of the cutting guide and inserts. Patient specific designs may optionally be fabricated using additive layer manufacturing, as described below.

In another aspect, the present disclosure relates to methods of manufacturing one or more components of the systems and kits contemplated by the present disclosure. In some embodiments, one or more of the frame, internal blocks, inserts, other accessories of the frame, and cutting guide may be formed using one or more of injection molding, forging or investment casting and rough machining. In other embodiments, additive layer manufacturing may be used to form one or more components of the instrumentation, systems and kits of the present disclosure. Examples of additive layer manufacturing techniques that may be utilized include Fused Deposition Modelling (“FDM”), Shape Deposition Manufacturing (“SDM”), Selective Laser Power Processing (“SLPP”), Direct Metal Laser Sintering (“DMLS”), Selective Laser Sintering (“SLS”), Selective Laser Melting (“SLM”), Selecting Heating Sintering (“SHS”), Electron Beam Melting (“EBM”), material jetting, binder jetting, or the like. Additional details of exemplary additive manufacturing methods are described in U.S. Pat. Nos. 7,537,664, 8,590,157, 8,728,387, 9,180,010 and 9,456,901, the disclosures of which are hereby incorporated by reference herein in their entireties.

In another aspect, the present disclosure relates to methods of retrieving and preparing implants such as allografts for implantation in a patient as part of a joint repair surgical procedure. Specific repair procedures that are envisioned include repair of the shoulder. It should be appreciated that although the example embodiments described below refer to a distal tibia as a donor bone, other donor bones are also contemplated for use as part of a method of the present disclosure. For example, a donor bone may be a distal radius, an ilium, a glenoid, a coracoid, a distal clavicle or a scapular spine.

In one embodiment, a method includes extraction of an allograft from a donor bone and implantation of the allograft in a shoulder of a patient, as shown through FIGS. 2-9. To prepare for extraction of an allograft from a donor bone, frame 10 is secured to a fixed support such as table 2 via support post 4, and first and second blocks 12, 22 are spaced apart so that a donor bone may be received in between first and second internal blocks 32, 42. In an optional initial step, a pair of inserts may be chosen by a user, and if inserts with different surface contours than first and second inserts 37, 47 secured to first and second internal blocks 32, 42, are deemed most appropriate for the procedure at issue, then such inserts may be swapped in and replace first and second inserts 37, 47, or may simply be the initial inserts secured to first and second internal blocks 32, 42. Each of first and second internal block 32, 42 is coupled to a respective first and second block 12, 22 through a tongue and groove connection, so attachment or removal of an internal block may be performed through sliding of the internal block onto or out of the respective block. Returning to the method, with a set of inserts decided upon, second block 22 is translated toward first block 12 until first and second inserts 37, 47 abut a surface of the donor bone, as shown in FIG. 3. In the depiction of this embodiment, the donor bone is a distal tibia 80. Frame 10 includes two separate mechanisms to control a relative position between first block 12 and second block 22. These mechanisms include a macro-translation mechanism and a micro-translation mechanism.

The macro-translation mechanism is in the form of simply using handles 19A-B, 29A-B (shown in FIG. 1) to hold frame 10 and push second block 22 toward first block 12. First and second blocks 12, 22 are coupled via rails 71A, 71B. In one variation, such rails 71A, 71B may be toothed so that translation may occur in increments. Further, frame 10 includes a pawl structure internal to a body of frame 10 with an exposed surface in the form of button 27. Button 27 may be actuated to release the pawl in order to pull second block 22 away from first block 12 so that distal tibia 80 may be adjusted relative to frame 10 or to remove frame 10 after the method is completed. As to the micro-translation mechanism, frame 10 includes an actuation shaft 78 passing into second block 22 and controlled by handle 76. Once first block 12 and second block 22 are at least in close contact with distal tibia 80, but not necessarily applying substantial pressure on tibia 80, the micro-translation mechanism may be used to fine tune the securement of distal tibia 80 between first and second internal blocks 32, 42 and to obtain an increased amount of compression against distal tibia 80. To operate the micro-translation mechanism, handle 76 is rotated to cause actuation shaft 78 to either advance or withdraw relative to end surface 23 of second block 22. When advanced, actuation shaft 78 contacts and/or engages second internal block 42 and causes internal block to move away from central inner surface 24 of second block 22. Using the macro- and micro-translation mechanisms, distal tibia 80 is securely set within and fixed to frame 10 to ensure tibia 80 does not shift or move when an allograft is cut from distal tibia 80.

With the donor bone, i.e., distal tibia 80, set in place within frame 10, a guide 100 is then attached to an end of distal tibia 80 as shown in FIG. 4. While the following steps describe performance of the method with guide 100, it should be appreciated that guides 200, 300, 400, 500 may also be used in the method in place of guide 100. To the extent the method may vary with these alternative guides, such method steps are described separately below. Guide 100 may be fixed in place onto distal tibia 80 via the placement of pins 191, 192 through openings in guide 100, as shown in FIG. 5. Pins 191, 192 may be drilled into distal tibia 80 through guide 100. Optionally, additional pins may be fixed in place through other holes in the guide. Also optionally, and prior to fixation of guide 100 onto distal tibia 80, a guide positioning tab 102 may be used to aid in the positioning of the guide. As shown in FIG. 4, guide positioning tab 102 may be inserted into first slit 112. Then, guide 100 may be moved toward a surface of distal tibia 80 until a tab portion (not shown) of guide positioning tab 102 presses against the bone surface. This provides an indication to a user that guide 100 is in an appropriate position for anchorage to distal tibia 80. Once the position of the guide 100 is finalized, guide positioning tab 102 may be removed from guide 100.

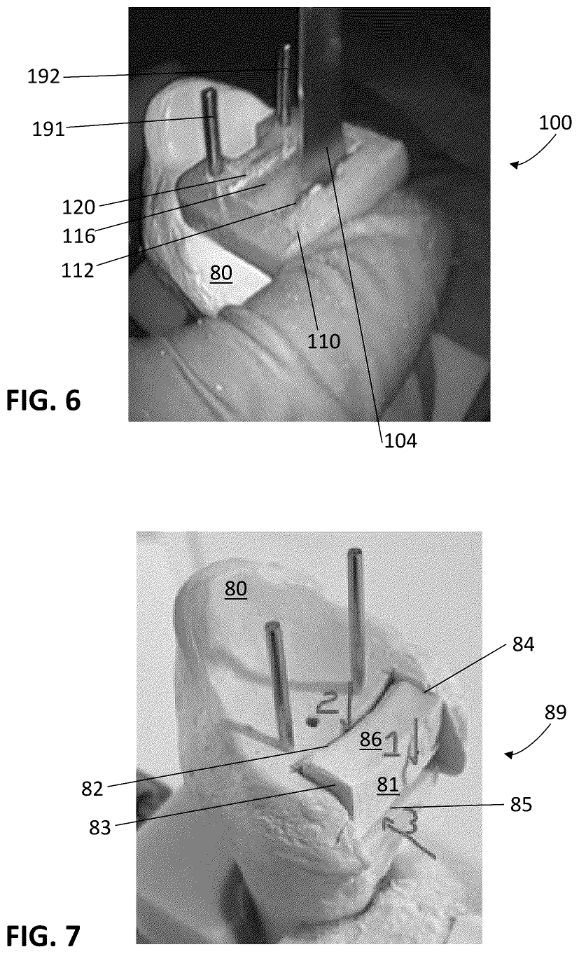

At this stage of the method, distal tibia 80 is held in place by frame 10, guide 100 is securely anchored to distal tibia 80, as shown in FIG. 5, and resection of distal tibia 80 may now be performed to retrieve an allograft. With guide 100 used to direct the cuts, a cut is made through first slit 112 and one slit from among second slit 116 and third slit 120 using a resection tool such as blade 104 shown in FIG. 6. In one example, blade 104 may be part of an oscillating cutting instrument. Whether a cut is made through second slit 116 or third slit 120 depends on an allograft thickness dimension sought, as shown in part in FIG. 6. These cuts through end plate 110 of guide 100 define surface 81 and angled inner surface 82 of allograft 89, shown fully cut in FIG. 7. A cut is also made through side slit 142 to define a lower surface 85 of allograft 89. Throughout the present disclosure, side slits may also be referred to as side plate slits. Similarly, slits on an end plate may be referred to as end plate slits. Guide 100 is then removed from distal tibia 80 and cutting may continue to make vertical cuts that are transverse to the cuts through the guide to define sides and width of allograft, as indicated by surfaces 83, 84 of fully cut allograft 89 shown in FIG. 7. Such vertical cuts may be performed based on measurement and marking (not shown) on distal tibia 80 performed by a user, and such preparatory steps may be performed prior to or after securement of guide 100 onto distal tibia 80. Following the completion of the tibial cuts, a standalone allograft 89 is created from the bone of distal tibia 80 interior to the cuts, as shown in FIG. 7. Allograft 89 may be trapezoidal in shape.

The method continues with the placement and securement of allograft 89 in a shoulder joint of the patient. An angled inner surface 82 of allograft 89 is positioned in an area of bone loss or in an area where bone is in an otherwise weakened state proximate a glenoid region 92, as shown in FIG. 8, allograft 89 being oriented such that surface 85 is inferior to surface 86. When allograft 89 is in a desired position on the bone of the patient, an anchoring pin 96 is installed through allograft 89 and into scapula 90, as shown in FIG. 9. From this point, with allograft 89 secured, any remaining steps may be completed to finalize the surgical procedure. In an alternative implantation configuration, allograft 89 may be implanted such that a depth of the allograft 89 extends from surface 81 to angled inner surface 82, a width of allograft extends across from surface 85 to surface 86, and a height of allograft 89 extends from surface 83 to surface 84. In such an alternative implanted configuration, allograft 89, when secured to a scapula, would appear as shown in FIG. 9 if viewed from above, i.e. in a superior-to-inferior direction.

In some embodiments, and as already noted, the shoulder repair method of the above-described embodiment may also be performed with a cutting guide other than guide 100. In these embodiments, the method may be performed in the same way as described above up to a step of securing a donor bone to frame 10, as shown in FIG. 3. From there, a different guide may be secured to the donor bone. These embodiments will be described with respect to distal tibia 80, although it should be appreciated that other donor bones may also be used. In the methods utilizing guides 200, 300, 400, 500, after the allograft is extracted from distal tibia 80, the remaining steps of the method may proceed as described above and shown in FIGS. 8 and 9.

When using guide 200, shown in FIG. 12A-14, guide 200 is pressed against distal tibia 80 so that contoured bone-facing surface 266 is flush with distal tibia 80, as shown in FIG. 13. Pins (not shown) may then be drilled or otherwise advanced through two or more central holes 281, 282, 283 in respective anchor guides 285, 286, 287 on guide 200 to secure guide 200 in place on distal tibia 80. Each opening may be formed within an anchor guide 285, 286, 287 with a sloped outer surface to direct a pin in a direction to avoid cutting planes and to ensure a secure anchorage of guide 200. Axes of such openings may be inward facing. In some embodiments, the method may be performed with pins that include a stop such as a circumferential flange or bulge along their length (not shown). Such stop may be included on the pins to control, i.e. limit an extent to which the pins extend into the bone to avoid overpenetration. Guide 200 is then used to direct a cutting tool through slits of guide 200 to cut out an allograft. There are several different allograft sizes that may be cut from distal tibia 80 based on the available slits on guide 200. Using the depicted embodiment as an example, a thickness of the allograft may be 7 mm or 10 mm. If the repair plan specifies a 7 mm thick allograft, then cuts are made through first slit 212 and second slit 216. If the repair plan specifies a 10 mm thick allograft, then cuts are made through first slit 212 and third slit 220. A location of these cut lines is shown in FIGS. 12A and 14. A height of the allograft may be 15 mm or 18 mm. If the repair specifies an allograft 15 mm in height, the cuts are made through upper cross-slits 246A, 246B. If the repair specifies an allograft 18 mm in height, the cuts are made through lower cross-slits 242A, 242B. A width of the allograft may be 18 mm or 23 mm. If the repair specifies an allograft 18 mm wide, then cuts are made through first inner slit 252 and second inner slit 254. Similarly, if the repair specifies an allograft 23 mm wide, then cuts are made through first outer slit 256 and second outer slit 258. With guide 200, all cuts to retrieve an allograft from the donor bone may be made using guide 200, without any separate measuring and marking on the bone. Additionally, it should be appreciated that use of a distal tibia or a distal radius as a donor bone for this method has been found to be advantageous in that the shape of such anatomy is well suited for retrieval of an allograft using a single guide as contemplated by the present disclosure.

Guide 300 may be anchored to distal tibia 80 in the same ways as described above for guide 200. When using guide 300, shown in FIGS. 15-17, cuts to establish a thickness of an allograft and a height of the allograft may be prepared in the same manner as described above for guide 200. To establish a width of allograft, cuts may be made along planar surfaces at respective first and second sides 362, 364 of side plate 340 on guide 300. To ensure a complete cut between first and second sides 362, 364, cuts made on side plate 340 through lower and upper cross-slits 342, 346, may be made by pressing a cutting tool against tapering first and second lateral sidewalls 343A, 343B to reach a wider extent of bone beneath outer surface 368. In one example, this may allow for a cut of up to 22 mm wide even when a width of slit on outer surface 368 is 19 mm. Thus, the tapered walls are advantageous in that they allow the cuts made through side plate 340 to reach side cuts made along first and second sides 362, 364. Further, while side plate 340 confers this advantage, it simultaneously has sufficient width at outer surface 368 to maintain structural integrity throughout a length of side plate 340.

When using guide 400, guide 400 is initially pressed against distal tibia 80 by pressing both first side plate 440 and second side plate 470 against respective outer surfaces of distal tibia 80, as shown in FIGS. 18-20. Once in position, anchors such as pins may be drilled into two or more holes (not shown) extending through guide 400. Although only one hole for an anchor is shown for guide 400, it should be appreciated that hole arrangements such as those shown for guides 200 and 300 may also be included for guide 400, among any others contemplated by the present disclosure. Once guide 400 is secured to distal tibia 80, a thickness and height of the allograft may be prepared in the same manner as described above for guide 200. For a width of the allograft, one slit from among inner slit 452 and outer slit 456 is cut through, depending on whether an 18 mm or 23 mm allograft width is sought. No additional cuts parallel to those along inner slit 452 or outer slit 456 are required when using this guide because the lower and upper cross-slits 442, 446 allow for a cut of distal tibia 80 that reaches or extends out of an outer surface of distal tibia 80 toward second side plate 470, and therefore advantageously there is no vertical cut required on that side of tibia 80.

In an alternative method, guide 400 is initially pressed against distal tibia 80 by pressing both first side plate 440 and second side plate 470 against respective outer surfaces of distal tibia 80, as shown in FIGS. 18-20. Once in position, anchors such as pins may be drilled into holes in guide 400 to anchor guide 400 to distal tibia 80. Anchor placement and an exact quantity and variation of anchor holes may vary as described for the previous embodiment or as otherwise contemplated for the methods of the present disclosure. Guide 400 is then used to direct a cutting tool to cut out an allograft from distal tibia 80. A width of the allograft is established through performance of a cut along outer side surface 482. A thickness of the allograft is established through performance of a cut along outer side surface 481. In some examples, a height of the allograft is established through performance of a cut along a slit in side plate 440, such as a 15 mm cut via upper cross-slit 446 or an 18 mm cut via lower cross-slit 442. In other examples, after cuts are completed via guidance of a cutting tool along outer side surfaces 481, 482, guide 400 is removed from distal tibia 80 and a remaining cut is made directly on the bone or the allograft is simply retrieved from a remainder of the bone manually. A third cut that is completed in this manner may be along a plane transverse to both the cutting plane along outer side surface 481 when guide 400 is attached to distal tibia 80 and the cutting plate along outer side surface 482 when guide 400 is attached to distal tibia 80.

In a variation of the above-described method that includes use of outer side surfaces 481, 482 as aids to cut bone with guide 400, such method may be performed in the same manner utilizing guide 400′ shown in FIG. 18A. With guide 400, a cut to define a height of the allograft may be completed after guide 400′ is removed from the bone. In yet another variation (not shown), guide 400′ may include a guide surface at an end of one of first side plate 440′ or second side plate 470′ remote from end plate 410′ so that a third cut may be made along such guide surface. Accordingly, in such variation, an allograft may be retrieved with three cuts using outer side surfaces of the guide 400.

Guide 500 may be anchored to distal tibia 80 using the same methods as contemplated for the other guides described above. When using guide 500, an initial cut may be a cut to define a width of the allograft along planar surfaces at first sides 532, 562, as shown in FIG. 21. Cuts to establish a thickness of an allograft and a height of the allograft may be prepared in the same manner as described above for guide 200. When guide 500 includes a second side plate to press against distal tibia 80, ends of slits 512, 516, 520, 542, 546 proximate second sides 534, 564 are either outside of or proximate to an outer surface of distal tibia 80, and therefore there is no vertical cut parallel to first sides 532, 562 required on that side of the bone.

Guide 600 may be anchored to distal tibia 80 using the same methods as contemplated for the other guides described above. When using guide 600, guide 600 may be positioned on a distal tibia by bringing convex apex 691 of bone-facing surface 636 on end plate 610 onto end trough 93 of tibia while also bringing bone-facing surface 666 of spacer 670 onto side trough 94 of distal tibia 80, as shown in FIGS. 27 and 28, for example. By positioning guide 600 on these respective surfaces, a predictable and desirable position of guide 600 relative to the bone is realized, from which guide 600 may be anchored to the bone via holes 681, 682, 683, for example. Once anchored, desired cuts may be made through the slits on guide 600.

A method of using a cutting guide to cut bone and prepare an allograft may be varied in many ways. In some embodiments, the method may include a step of selecting a guide type or size prior to securing the guide to distal tibia 80. The guide may be one of guide 100, 200, 300, 400, 500, 600 or any other guide contemplated by the present disclosure. In some examples of these embodiments, the selection step may be part of a pre-operative planning process as described elsewhere in the present disclosure. In any of the above-described embodiments, the cuts may be performed in any desired order, unless explicitly stated to the contrary.

In some embodiments of the method, a further step may be performed to prepare a segment of soft tissue, i.e., through flattening, for use in the shoulder repair, as shown via FIGS. 10 and 11. In these embodiments, either prior to or after using frame 10 to retrieve an allograft, frame 10 is repositioned so that support post 4 is received in outside surface 13 of first block 12, and secured to a fixed support such as table 2 shown in FIG. 11. In this arrangement, central longitudinal axis 8 along the body of frame 10 is oriented generally perpendicular to table 2, in contrast to the configuration of frame 10 for allograft retrieval, where central longitudinal axis 8 along the body of frame 10 is generally parallel to table 2. With frame 10 secured, third and fourth internal blocks 52, 62, shown in FIG. 10, are slid onto frame 10 as shown in FIG. 11. Of course, if other internal blocks are already on frame 10, those are removed first. A segment of soft tissue (not shown), such as a bicep tendon may then be placed into cavity 55 of third internal block 52. At this juncture, frame 10 is set up to flatten the soft tissue. The macro-translation mechanism and the micro-translation mechanism may then be used to position third and fourth internal blocks 52, 62 in order to advance protrusion 65 of fourth internal block 62 into cavity 55 of third internal block 52. The translation mechanisms may be operated in the same way as described above where frame 10 was fitted with first and second internal blocks 32, 42. Fourth internal block 62 is advanced toward third internal block 52 until protrusion 65 makes contact with and begins to compress and flatten the soft tissue within cavity 55. A current thickness of the soft tissue may be monitored through a position of flange 68 relative to third internal block 52 so that a user can fine tune an extent of compression of the soft tissue. To ensure the soft tissue does not return to its preexisting condition after being pressed between protrusion 65 and cavity 55, an advanced position of protrusion 65 within cavity 55 that involves application of pressure to the soft tissue may be maintained for a duration of time as desired. When the segment soft tissue is flattened to a desired extent, it may be employed as appropriate as an additional part of the shoulder repair.

In other embodiments, a method may include only some steps of the above-described methods of shoulder repair. For example, a method may only involve steps to extract an allograft from a donor bone, as described above. In another example, a method may only involve steps to flatten a soft tissue segment, as described above.

In still further embodiments, the above-described methods may be performed using frame 710 to clamp a donor bone, e.g., a cadaver, so that when clamped, an allograft may be extracted from the donor bone via cuts made through a cutting guide. Features of frame 710 are shown in FIGS. 29-33. The method may begin by securing frame 710 onto a fixed surface, such as a table. In one example, post 704 is inserted into a receptacle on the fixed surface while extension 707 of sleeve 706 is pulled to fit extension 707 over the fixed surface edge. This way, the extension compresses against the fixed surface edge when released causing the frame 710 to become fixed to the fixed surface.

Either before or after frame 710 is mounted on the fixed surface, first and second inner members 732, 742 are inserted into respective first and second outer members 712, 722. First inner member 732 is inserted from above first outer member 712 so that first inner member 732 fits in between protrusions 718A, 718B. Advancement of first inner member 732 continues until first inner member 732 is proximate to or contacts ledge 715, and at such juncture, spring-loaded grippers 719A, 719B, shown in FIG. 32, expand against first inner member 732 to hold first inner member 732 against protrusions 718A, 718B, thereby stabilizing first inner member 732. An inserted position of first inner member 732 is shown in FIGS. 29, 31 and 33. As to second inner member 742, a user first checks to ensure that actuation shaft is sufficiently withdrawn from the recessed region of second outer member 722, then second inner member 742 is advanced in between protrusions 728A, 728B with undercut 749 within recess 748 advancing over head 779 at the end of actuation shaft 778. Similar to first inner member 732, second inner member 742 is advanced until such inner member is proximate to or contacts ledge 726, as shown in FIGS. 29, 31 and 33. And, for second inner member 742, spring loaded grippers 729A, 729B press against sides of second inner member 742 to further stabilize its position. In some variations, one or both of first and second inner members 732, 742 includes a step on an outer surface so that when fully inserted into an applicable outer member, the inner member is also held in place against withdrawal from the outer member by a spring loaded member pressing against the inner member immediately above the step.

Upon assembly of the inner members within the respective outer members, as shown in FIG. 29, for example, first and second outer members 712, 722 are separated, if not already separated, sufficiently to create space for a donor bone to be received in between first and second inner members 732, 742. In the depicted version of frame 710, buttons of first and second pawls 727A, 727B may be pressed to allow second outer member 722 to be drawn away from first outer member 712. Then, a donor bone, such as distal tibia 80 (not shown in FIGS. 29-33), is placed in between first and second inner members 732, 742.

To effectuate a clamping of the donor bone by inserts, such as first and second gripping members 737, 747 of frame 710, frame 710 includes macro- and micro-translation mechanisms, similar to frame 10. Macro-translation mechanism, already referenced above, includes a pair of rails 771A, 771B complemented by respective first and second pawls 727A, 727B, as shown in FIG. 29, for example. As depicted, each rail includes a plurality of teeth so that when the first and second pawls 727A, 727B are in a neutral, non-actuated state, the pawl grips the respective rail and the relative position of first and second outer members 712, 722 is stable and fixed. To bring first and second outer members 712, 722 closer together, the buttons of first and second pawls 727A, 727B are pressed down and then the outer members are pushed toward each other. To obtain further clamping, e.g., any remaining clamping needed to apply pressures against a donor bone such as distal tibia 80, a micro-translation mechanism is used. In frame 710, micro-translation mechanism includes actuation shaft 778 and its associated components, best shown in FIG. 33. To control a position of second inner member 742 relative to second outer member 722, handle 776 is rotated, thereby causing head 779 at an opposite end of actuation shaft 778 to become either further or closer to central inner surface 724 of the recessed portion of second outer member 722, depending on the direction of rotation of knob. As shown, clockwise rotation of handle 776 causes second inner member 742 to move away from central inner surface 724, thereby causing second inner member 742, inclusive of second gripping member 747, to move closer to and ultimately press against the donor bone with opposing first gripping member 737 on an opposite side of donor bone. Such fine tuning may be used to obtain a desired amount of clamping on the donor bone.

Once a donor bone is clamped within frame 710, the method may continue with the attachment and use of any cutting guide contemplated by the present disclosure to extract an allograft from the donor bone. Further, as with frame 10, frame 710 may also be adapted to receive alternative first and second inner members, either with shapes for use with different bone types, or for use in compressing soft tissue, similar to the third and fourth inner blocks 52, 62 shown in FIGS. 10 and 11.

In still further embodiments, a method of preparing a segment of soft tissue may be performed using a frame in conjunction with first and second inner members 1052, 1062. Such preparation according to these methods includes flattening the soft tissue through the application of force against the soft tissue to compress it. In one example, such method may be performed with frame 1010 shown in FIG. 36 and inner members 1052, 1062, shown in FIGS. 36-38, attached to support components of frame 1010. This method may be performed as a standalone method or as a part of a larger method of surgery.

In this method, frame 1010 shown in FIG. 36 may be used and soft tissue (not shown) is positioned into cavity 1055 of first inner member 1052. The soft tissue may be a biceps tendon, for example. To keep the soft tissue within cavity 1055, frame 1010 may be oriented so that an opening of cavity 1055 faces away from the ground. If frame 1010 is oriented so that cavity 1055 faces sideways relative to the ground, temporary fixation may optionally be used to hold the soft tissue within cavity 1055. At this juncture, frame 1010 is set up to flatten the soft tissue. The actuation shaft 1078 may be rotated, e.g., via use of handle 1076, to bring first inner member 1052 toward second inner member 1062. During such process, as first inner member 1052 becomes closer to second inner member 1062, protrusion 1065 moves into cavity 1055 and with continued rotation of handle 1076, the soft tissue comes into contact with both cavity surface 1055A and protrusion surface 1065A. Further rotation of handle causes the soft tissue to compress and flatten within cavity 1055. During this process, a real time compression of the soft tissue, e.g., evidenced by reduction in thickness and increase in surface area, may be evaluated through monitoring of the soft tissue through windows 1031A-D. Specifically, after observing an initial position of the soft tissue within one or more windows from among windows 1031A-D, i.e., a position when the pair of surfaces 1055A, 1065A first come into contact with the soft tissue, a user may monitor creep of the soft tissue using the markers of the sets of size markers 1041A-D, 1042A-D, as shown in FIG. 37. Through a comparison between the initial position and a changed position, a change in the overall dimensions of the soft tissue may be observed and measured, and an extent of compression may be derived from such measurement.

The application of compression to the soft tissue as part of the performance of the method may be performed in a variety of ways. In some examples, actuation shaft 1078 may be rotated at a continuous rate such that once soft tissue is pressed between surfaces 1055A, 1065A, compression against the soft tissue increases in a continuous manner. In other examples, actuation shaft 1078 may be operated in a cyclical manner. For instance, actuation shaft 1078 may be operated to advance first inner member 1052 to a first minimum spacing from second inner member 1062, then reversed, then advanced again but this time to a second minimum spacing less than the first minimum spacing.

To ensure the soft tissue does not return to its preexisting condition after being compressed between protrusion 1065 and cavity 1055, the method may be performed such that once a desired compression position of protrusion 1065 within cavity 1055 is reached, such position may be maintained for a duration of time as deemed appropriate. Once the flattening process is completed and the segment of soft tissue is flattened to a desired extent, such soft tissue may be employed as appropriate as an additional part of a shoulder repair procedure.

While the above method is described with the use of frame 1010, it should be appreciated that the method of flattening soft tissue using first and second inner members 1052, 1062 may be performed with other frames as contemplated by the present disclosure.

While the methods described in the present disclosure are performed manually, other embodiments may employ robots to aid in the use of the various parts of the instruments, systems and kits contemplated by the present disclosure. In one non-limiting example, a robot may be programmed to control operation of the frame in its various contemplated uses. And, in other examples, a robot may be used to perform cuts using one or more of the guides contemplated by the present disclosure.

The present disclosure provides improved systems and kits for use in the preparation of allografts for a shoulder surgery. Advantages include that only one cutting guide is needed to prepare a hard tissue allograft and that components can easily be substituted to account for different types or sizes of donor bone to ensure a good fit for allograft retrieval. Additionally, a single frame system may be used for gripping a donor bone and for flattening a soft tissue. Moreover, the methods of using the systems and kits are straightforward and do not require adjustment, alignment and/or calibration between the frame and the cutting guide thereby simplifying the method of retrieving an allograft.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A guide for retrieving donor bone material for implantation in a patient comprising:
an end plate having an outer surface and a bone-facing surface opposite the outer surface, the end plate including a first guide surface; and
a side plate extending at an angle from the end plate, the side plate including a second guide surface.

2. The guide of claim 1, wherein the bone-facing surface of the end plate includes an end plate central protrusion that is positionable within a first trough on an end surface of the donor bone.

3. The guide of claim 2, wherein the side plate includes a bone-facing surface with a side plate central protrusion thereon, the side plate central protrusion being aligned with and offset from opposing outer side surfaces of the side plate and being sized to be positionable within a second trough on a side surface of the donor bone when the end plate central protrusion of the end plate is positioned within the first trough on the end surface of the donor bone.

4. The guide of claim 1, wherein the end plate and the side plate are formed together monolithically.

5. The guide of claim 1, wherein the first guide surface defines part of a first end plate slit passing through the outer and bone-facing surfaces of the end plate.

6. The guide of claim 5, further comprising a second end plate slit spaced apart from the first end plate slit, the second end plate slit passing through the outer and bone-facing surfaces of the end plate and defining a cutting plane at an angle relative to a cutting plane of the first end plate slit.

7. The guide of claim 6, wherein the second guide surface is a portion of an outer side surface of the side plate such that cutting through the first and second end plate slits defines a thickness dimension of an allograft, cutting through a side plate slit of the side plate defines a height dimension of the allograft, and cutting along the outer side surface at least partially defines a width dimension of the allograft.

8. The guide of claim 6, wherein the side plate includes opposing outer side surfaces extending from the end plate to a free end of the side plate, and the second guide surface defines part of a first side plate slit in between the opposing outer side surfaces, the side plate further comprising a second side plate slit such that a cutting plane of the second side plate slit is orthogonal to a cutting plane of the first side plate slit.

9. The guide of claim 8, wherein the end plate defines an opening configured to receive a first bone anchor and the side plate defines an opening configured to receive a second bone anchor.

10. The guide of claim 1, wherein the first guide surface is an outer side surface of the end plate.

11. The guide of claim 1, wherein the side plate includes opposing outer side surfaces extending from the end plate to a free end of the side plate, and the second guide surface defines part of a side plate slit in between the opposing outer side surfaces.

12. The guide of claim 1, wherein the second guide surface is an outer side surface of the side plate.

13. The guide of claim 1, wherein the side plate is a first side plate and the guide further comprises a second side plate extending from the end plate and from the first side plate, the second side plate being oriented at an angle relative to the end plate and the first side plate.

14. The guide of claim 1, wherein at least one of the end plate and the side plate is configured to be attached directly to the donor bone.

15. The guide of claim 1, wherein the guide defines one or more openings, each of the one or more openings being configured to receive one of an anchor or a pin to fix the guide to the donor bone.

16. A method of retrieving an allograft for implantation in a patient comprising:
securing a cutting guide to a donor bone such that an end plate of the cutting guide faces an end surface of the donor bone and a side plate of the cutting guide that extends from the end plate faces a side surface of the donor bone;
forming a first bone cut through the end surface of the donor bone using a cutting tool positioned along a first guide surface of the end plate;
forming a second bone cut through the side surface of the donor bone using the cutting tool positioned along a second guide surface of the side plate; and
forming a third bone cut through the donor bone, the third bone cut being at an angle relative to each of the first and second bone cuts,
wherein a portion of the donor bone defined by the first, second and third bone cuts encompasses a bone segment to be implanted into a joint of the patient.

17. The method of claim 16, further comprising forming a fourth bone cut through the end surface of the donor bone using the cutting tool positioned through a plate slit in the end plate.

18. The method of claim 16, further comprising forming a fourth bone cut through the side surface of the donor bone using the cutting tool positioned through a plate slit in the side plate.

19. The method of claim 16, wherein the first guide surface defines part of a plate slit in the end plate and forming the first bone cut includes using the cutting tool positioned through the plate slit.

20. A system for retrieving donor bone material for implantation in a patient, the system comprising:
a frame comprising:
a first member including a first gripping surface; and
a second member movably coupled to the first member, the second member including a second gripping surface, wherein the first member and the second member are configured to clamp the donor bone; and
a guide configured for attachment to a donor bone while the donor bone is held fixed by the first and second gripping surfaces, the guide comprising:
an end plate having an outer surface and a bone-facing surface opposite the outer surface, the end plate including a first guide surface; and
a side plate extending at an angle from the end plate, the side plate including a second guide surface,
wherein when the first and second gripping surfaces hold the donor bone fixed relative to the frame and the guide is fixed relative to the donor bone, a portion of the donor bone separates an entirety of the frame from an entirety of the guide, and the donor bone remains stable while cuts are made along the first guide surface and the second guide surface.
